SEQUENCE LISTING (16 sequences)

SEQ ID NO: 1 - protein, 595 aa, Unknown
  Populus canescens
MATELLCLHRPISLTHKLFRNPLPKVIQATPLTLKLRCSVSTENVSFTETETEARRSANYEPNSWDYDFLLSSDTDESIEVYKDKAKKLEAEVRREINNEKAEFLTLLELIDNVQRLGLGYRFESDIRRALDRFVSSGGFDGVTKTSLHATALSFRLLRQHGFEVSQEAFSGFKDQNGNFLENLKEDTKAILSLYEASFLALEGENILDEARVFAISHLKELSEEKIGKELAEQVNHALELPLHRRTQRLEAVWSIEAYRKKEDANQVLLELAILDYNMIQSVYQRDLRETSRWWRRVGLATKLHFAKDRLIESFYWAVGVAFEPQYSDCRNSVAKMFSFVTIIDDIYDVYGTLDELELFTDAVERWDVNAINDLPDYMKLCFLALYNTINEIAYDNLKDKGENILPYLTKAWADLCNAFLQEAKWLYNKSTPTFDDYFGNAWKSSSGPLQLIFAYFAVVQNIKKEEIENLQKYHDIISRPSHIFRLCNDLASASAEIARGETANSVSCYMRTKGISEELATESVMNLIDETCKKMNKEKLGGSLFAKPFVETAINLARQSHCTYHNGDAHTSPDELTRKRVLSVITEPILPFER

SEQ ID NO: 2 - protein, 778 aa, Solanum pennellii
MIVGYRSTIITLSHPKLGNGKTISSNAIFQRSCRVRCSHSTTSSMNGFEDARDRIRESFGKLELSPSSYDTAWVAMVPSKHSLNEPCFPQCLDWIIENQREDGSWGLNPTHPLLLKDSLSSTLACLLALTKWRVGDEQIKRGLGFIETYGWAVDNKDQISPLGFEVIFSSMIKSAEKLDLNLPLNLHLVNLVNCKRDSTIKRNVEYMGEGVGELCDWKEIIKLHQRQNGSLFDSPATTAAALIYHQHDQKCNQYLNSILKQHKNWVPTMYPTKIHSLLCLVDTLQNLGVHRHFKSEIKKALDEIYRLWQQKNEQIFSNVTHCAMAFRLLRMSYYDVSSDELAEFVDEEHFFTTSGKYTSHVEILELHKASQLAIDHEKDDILDKINNWTRTFMEQKLLNNGFIDRMSKKEVELALKKFYTTSDLAENRRYIKSYEENNFKILKAAYRSPNINNKDLLAFSIHEFELCQAQHREELQQLRRWFEDYRLDQLGLAERYIHATYLFGVTIIPEPELSDARLMNAKYIMLLTIVDEYFESFASKDECLNIIELVERWDDYASVGYKSEKVKVFFSTFYKSIEELATIAEIKQGRSVKNHLINLWLEVMKLMLMEQVEWWTSKTIPSIEEYLCVTSITFGSRLLLLTIQYFLGIKISKDLLESDEICGLCNCTGRVMRILNDLQDSKREQKGVSINLVTLLMKSISEEEAIMKMKEILEMNRRELLKMVLVQKKGSQLPQLCKDIFWRTSKWTHFTYSQTDGFRIEEEMKNHIDEVFYKPLNH

SEQ ID NO: 3 - protein, 630 aa, Abies grandis
MALVSSAPKSCLHKSLIRSTHHELKPLRRTIPTLGMCRRGKSFTPSVSMSLTTAVSDDGLQRRIGDYHSNLWDDDFIQSLSTPYGEPSYRERAEKLIGEVKEMFNSMPSEDGESMSPLNDLIERLWMVDSVERLGIDRHFKKEIKSALDYVYSYWNEKGIGCGRDSVFPDVNSTASGFRTLRLHGYSVSSEVLKVFQDQNGQFAFSPSTKERDIRTVLNLYRASFIAFPGEKVMEEAEIFSSRYLKEAVQKIPVSSLSQEIDYTLEYGWHTNMPRLETRNYLDVFGHPTSPWLKKKRTQYLDSEKLLELAKLEFNIFHSLQQKELQYLSRWWIHSGLPELTFGRHRHVEYYTLSSCIATEPKHSAFRLGFAKTCHLITVLDDIYDTFGTMDEIELFNEAVRRWNPSEKERLPEYMKEIYMALYEALTDMAREAEKTQGRDTLNYARKAWEVYLDSYTQEAKWIASGYLPTFEEYLENAKVSSGHRAAALTPLLTLDVPLPDDVLKGIDFPSRFNDLASSFLRLRGDTRCYKADRDRGEEASSISCYMKDNPGLTEEDALNHINAMINDIIKELNWELLKPDSNIPMTARKHAYEITRAFHQLYKYRDGFSVATQETKSLVRRTVLEPVPL

SEQ ID NO: 4 - protein, 574 aa, Artemisia annua
MSTLPISSVSFSSSTSPLVVDDKVSTKPDVIRHTMNFNASIWGDQFLTYDEPEDLVMKKQLVEELKEEVKKELITIKGSNEPMQHVKLIELIDAVQRLGIAYHFEEEIEEALQHIHVTYGEQWVDKENLQSISLWFRLLRQQGFNVSSGVFKDFMDEKGKFKESLCNDAQGILALYEAAFMRVEDETILDNALEFTKVHLDIIAKDPSCDSSLRTQIHQALKQPLRRRLARIEALHYMPIYQQETSHDEVLLKLAKLDFSVLQSMHKKELSHICKWWKDLDLQNKLPYVRDRVVEGYFWILSIYYEPQHARTRMFLMKTCMWLVVLDDTFDNYGTYEELEIFTQAVERWSISCLDMLPEYMKLIYQELVNLHVEMEESLEKEGKTYQIHYVKEMAKELVRNYLVEARWLKEGYMPTLEEYMSVSMVTGTYGLMIARSYVGRGDIVTEDTFKWVSSYPPIIKASCVIVRLMDDIVSHKEEQERGHVASSIECYSKESGASEEEACEYISRKVEDAWKVINRESLRPTAVPFPLLMPAINLARMCEVLYSVNDGFTHAEGDMKSYMKSFFVHPMVV

SEQ ID NO: 5 - DNA, 99 bp, Artificial
  Linker
agctggcatgcagagtttaaacccagggcgcgccacgcgtccttcaattgccgcgatcgttaattaaccgccacctaggcctctgcggccgcactagtc

SEQ ID NO: 6 - DNA, 99 bp, Artificial
  Linker
gatcgactagtgcggccgcagaggcctaggtggcggttaattaacgatcgcggcaattgaaggacgcgtggcgcgccctgggtttaaactctgcatgcc

SEQ ID NO: 7 - DNA, 964 bp, Unknown
  DNA fragment for Puromycin resistance
gcatgcatcttctattttcatcttaattttatatattaaaggctaatttttcaatattttatcgcaaactattgtgttaattacatcaaatatataaacttttctatttaaagctaatgaaaaagtgaatatatacacatagagtaatgttatgatgtatatatcaaaaaaataggagtgattcctatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagttaattactaatttattattaatttattattagattgggcaaaatagtaaaagaaaactaaaggaaacctaatatggtttcctttttttatatatttttaaaaattgattatattatatgtaaacaattataattaatgttaatcgaattcaatttttatttaataaacggcgcgcc

SEQ ID NO: 8 - DNA, 2918 bp, Artificial
  DNA fragment encoding IDI-IspS
ttaattaaatgatttttaaaaaaatacatgaattcatctagcggagaacacaaaagatttaagtaccttctaaacgaatgagatttcattgggaaagtggacacttaagaacgacgcggtacttgatttattgagtgcaaaagcactcgattaggtgaccagtcccaaagtgattttaataaattaaggaggaaattaaaatgactgccgacaacaatagtatgccccatggtgcagtatctagttacgccaaattagtgcaaaaccaaacacctgaagacattttggaagagtttcctgaaattattccattacaacaaagacctaatacccgatctagtgagacgtcaaatgacgaaagcggagaaacatgtttttctggtcatgatgaggagcaaattaagttaatgaatgaaaattgtattgttttggattgggacgataatgctattggtgccggtaccaagaaagtttgtcatttaatggaaaatattgaaaagggtttactacatcgtgcattctccgtctttattttcaatgaacaaggtgaattacttttacaacaaagagccactgaaaaaataactttccctgatctttggactaacacatgctgctctcatccactatgtattgatgacgaattaggtttgaagggtaagctagacgataagattaagggcgctattactgcggcggtgagaaaactagatcatgaattaggtattccagaagatgaaactaagacaaggggtaagtttcactttttaaacagaatccattacatggcaccaagcaatgaaccatggggtgaacatgaaattgattacatcctattttataagatcaacgctaaagaaaacttgactgtcaacccaaacgtcaatgaagttagagacttcaaatgggtttcaccaaatgatttgaaaactatgtttgctgacccaagttacaagtttacgccttggtttaagattatttgcgagaattacttattcaactggtgggagcaattagatgacctttctgaagtggaaaatgacaggcaaattcatagaatgctataaacaaattagttaaaaggtctaaagatggaagccagacggtctgccaattatgaaccaaatagctgggattatgattttttgctgtcttcagacactgacgaatcgattgaagtatacaaagacaaggccaaaaagctggaggctgaggtgagaagagagattaacaatgaaaaggcagagtttttgactctgcttgaactgatagataatgtccaaaggttaggattgggttaccggttcgagagtgacataaggagagccctcgacagatttgtttcttcaggaggatttgatggtgttacaaaaactagccttcatgctactgctcttagcttcaggcttctcagacagcatggctttgaggtctctcaagaagcgttcagtggattcaaggatcaaaatggcaatttcttggaaaaccttaaggaggacaccaaggcaatactaagcctatatgaagcttcatttcttgcattagaaggagaaaatatcttggatgaggccagggtgtttgcaatatcacatctaaaagagctcagcgaagaaaagattggaaaagagctggccgaacaggtgaatcatgcattggagcttccattgcatcgcaggacgcaaagactagaagctgtttggagtattgaagcataccgtaaaaaggaagatgcaaatcaagtactgctagaacttgctatattggactacaacatgattcaatcagtataccaaagagatcttcgcgagacatcaaggtggtggaggcgagtgggtcttgcaacaaagttgcattttgctaaagacaggttaattgaaagcttttactgggcagttggagttgcgttcgaacctcaatacagtgattgccgtaattcagtagcaaaaatgttttcatttgtaacaatcattgatgatatctatgatgtttatggtactctggatgagctggagctatttacagatgctgttgagagatgggatgttaacgccatcaatgatcttccggattatatgaagctctgcttcctagctctctacaacactatcaatgagatagcttatgacaatctgaaggacaagggggaaaacattcttccatacctaacaaaagcgtgggcagatttatgcaatgcattcctacaagaagcaaaatggctgtacaataagtccacaccaacatttgatgactatttcggaaatgcatggaaatcatcctcagggcctcttcaactaatttttgcctactttgccgtggttcaaaacatcaagaaagaggaaattgaaaacttacaaaagtatcatgatatcatcagtaggccttcccacatctttcgtctttgcaacgacctggcttcagcatcggctgagatagcgagaggtgaaactgcgaattccgtatcctgctacatgcgtacaaaaggcatttctgaggaacttgctactgaatccgtaatgaatttgatcgacgaaacctgtaaaaagatgaacaaagaaaagcttggtggctctttgtttgcaaaaccttttgtcgaaacagctattaaccttgcacggcaatcccattgcacttatcataacggagatgcgcatacttcaccagacgagctaactaggaaacgtgtcctgtcagtaatcacagagcctattctaccctttgagagataaactcaataaattaaacaacttaaaacagagcaggccttcgggcctgctttttctttctttcccgagtcccgcctcgggagggcggtgtcctttccgccccgaattgcgcatcgggagcacgcggtccttttcgctcgctttgctctctcaagaggactaatttacaaaattacatattgagagcgcggccgc

SEQ ID NO: 9 - DNA, 11613 bp, Artificial
  Vector pACPmcr-IDI-IspS
gaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagcttgcatgcatcttctattttcatcttaattttatatattaaaggctaatttttcaatattttatcgcaaactattgtgttaattacatcaaatatataaacttttctatttaaagctaatgaaaaagtgaatatatacacatagagtaatgttatgatgtatatatcaaaaaaataggagtgattcctatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagttaattactaatttattattaatttattattagattgggcaaaatagtaaaagaaaactaaaggaaacctaatatggtttcctttttttatatatttttaaaaattgattatattatatgtaaacaattataattaatgttaatcgaattcaatttttatttaataaacggcgcgccacgcgtccttcaattgccgcgatcgttaattaaatgatttttaaaaaaatacatgaattcatctagcggagaacacaaaagatttaagtaccttctaaacgaatgagatttcattgggaaagtggacacttaagaacgacgcggtacttgatttattgagtgcaaaagcactcgattaggtgaccagtcccaaagtgattttaataaattaaggaggaaattaaaatgactgccgacaacaatagtatgccccatggtgcagtatctagttacgccaaattagtgcaaaaccaaacacctgaagacattttggaagagtttcctgaaattattccattacaacaaagacctaatacccgatctagtgagacgtcaaatgacgaaagcggagaaacatgtttttctggtcatgatgaggagcaaattaagttaatgaatgaaaattgtattgttttggattgggacgataatgctattggtgccggtaccaagaaagtttgtcatttaatggaaaatattgaaaagggtttactacatcgtgcattctccgtctttattttcaatgaacaaggtgaattacttttacaacaaagagccactgaaaaaataactttccctgatctttggactaacacatgctgctctcatccactatgtattgatgacgaattaggtttgaagggtaagctagacgataagattaagggcgctattactgcggcggtgagaaaactagatcatgaattaggtattccagaagatgaaactaagacaaggggtaagtttcactttttaaacagaatccattacatggcaccaagcaatgaaccatggggtgaacatgaaattgattacatcctattttataagatcaacgctaaagaaaacttgactgtcaacccaaacgtcaatgaagttagagacttcaaatgggtttcaccaaatgatttgaaaactatgtttgctgacccaagttacaagtttacgccttggtttaagattatttgcgagaattacttattcaactggtgggagcaattagatgacctttctgaagtggaaaatgacaggcaaattcatagaatgctataaacaaattagttaaaaggtctaaagatggaagccagacggtctgccaattatgaaccaaatagctgggattatgattttttgctgtcttcagacactgacgaatcgattgaagtatacaaagacaaggccaaaaagctggaggctgaggtgagaagagagattaacaatgaaaaggcagagtttttgactctgcttgaactgatagataatgtccaaaggttaggattgggttaccggttcgagagtgacataaggagagccctcgacagatttgtttcttcaggaggatttgatggtgttacaaaaactagccttcatgctactgctcttagcttcaggcttctcagacagcatggctttgaggtctctcaagaagcgttcagtggattcaaggatcaaaatggcaatttcttggaaaaccttaaggaggacaccaaggcaatactaagcctatatgaagcttcatttcttgcattagaaggagaaaatatcttggatgaggccagggtgtttgcaatatcacatctaaaagagctcagcgaagaaaagattggaaaagagctggccgaacaggtgaatcatgcattggagcttccattgcatcgcaggacgcaaagactagaagctgtttggagtattgaagcataccgtaaaaaggaagatgcaaatcaagtactgctagaacttgctatattggactacaacatgattcaatcagtataccaaagagatcttcgcgagacatcaaggtggtggaggcgagtgggtcttgcaacaaagttgcattttgctaaagacaggttaattgaaagcttttactgggcagttggagttgcgttcgaacctcaatacagtgattgccgtaattcagtagcaaaaatgttttcatttgtaacaatcattgatgatatctatgatgtttatggtactctggatgagctggagctatttacagatgctgttgagagatgggatgttaacgccatcaatgatcttccggattatatgaagctctgcttcctagctctctacaacactatcaatgagatagcttatgacaatctgaaggacaagggggaaaacattcttccatacctaacaaaagcgtgggcagatttatgcaatgcattcctacaagaagcaaaatggctgtacaataagtccacaccaacatttgatgactatttcggaaatgcatggaaatcatcctcagggcctcttcaactaatttttgcctactttgccgtggttcaaaacatcaagaaagaggaaattgaaaacttacaaaagtatcatgatatcatcagtaggccttcccacatctttcgtctttgcaacgacctggcttcagcatcggctgagatagcgagaggtgaaactgcgaattccgtatcctgctacatgcgtacaaaaggcatttctgaggaacttgctactgaatccgtaatgaatttgatcgacgaaacctgtaaaaagatgaacaaagaaaagcttggtggctctttgtttgcaaaaccttttgtcgaaacagctattaaccttgcacggcaatcccattgcacttatcataacggagatgcgcatacttcaccagacgagctaactaggaaacgtgtcctgtcagtaatcacagagcctattctaccctttgagagataaactcaataaattaaacaacttaaaacagagcaggccttcgggcctgctttttctttctttcccgagtcccgcctcgggagggcggtgtcctttccgccccgaattgcgcatcgggagcacgcggtccttttcgctcgctttgctctctcaagaggactaatttacaaaattacatattgagagcgcggccgcactagtaagttatagaaatgtcatccgataccgaaaaagaaaacggaataaatcagtaacaaatcaaaaaaacaaaactataacatttaatcttcgtaaaatcaatatcaatcgaccatacacaaaacatgttcaaataaaatacatgggtgtacatatgtcaaccgacattgaaaaagaaaacggaataaatcagtaacaaatcaaaaaaacaaaaccataacatttctataacattccatagagtaaaaatcaacatcgaataaccttacacaaaccatacataaaccatacataaaaatacacatatgtcaaccgttaccgtaaaaagattacttaaataattttatactcacctcttattttttgtgttttttattatccagggcaacctttcccagggagaaattcttccttgcagttgcaggcaagaaccaggactttcttccagggcatactttcccagggagaaattcttccttgcagctgcaggcaagaaccaggactttcccccaggaccaggacctcaaggtctgcatccagaggaagaccttcccagggcgccaagatcccgcagattatggaacattatttcccgggaaataattcagggcttaatgcccaactgaggaatacattttaaataagcttctcttagttcatccttttctatgtggtcgtacacgtctattgcttcgcctctcgaatctccccttaattcctgaatgaatgtcctttccatacctgaacgtcgaagccatgtagtgaaccaatgccggcagcaatgcggagtaaaatgttcttcaagtctcccctcggggttatggagtccgatagaggaagcggcagatgtgacaatatcgtacactaaatttctttgtacacggtgaccgttttgtctgcctatgaataatgctttttctttatcatgcctcgtgttaaggtacgcttccaggaaggccttagtctcaaggtcaaagaatacgatacgatttgaccttttagctgtcttcttgagtctcaccgtacacttttccagatctatgtcagctcgatcaagagtaattaattcatttctcctgatgccagttttagctaaaaagattatcaatgcttgatacatggggtaacgtggtactttgacaagctttgccatgtcttcaatggtaatcaattgccttgtttccggagtatggtattttttgtagcttcttagatagcgttttctgaacgtgggaatgttgtttctctcgattatatgcttgaattcaaggaagtcaaaaaaggtactcagtgaagcaaagtaattgttacaggttgcgagtgtgtactgcctttcatctcttatgtgaacaaggaacccttcaagctccggaaaccctgcattcactccacacatttgaaggaaaaacttagcatgacttacgtatccttctattgttcttttagagtaaccccttccgatacaatcagaagtgaataaacttataagtctttcattatcaagcggttcatagataccagttagctgaatagctgccatttcacccataaatcaataaaagaagtatgtaaataaatactttgtgcatagatgtcatttaaacacaaagtataagtacatctaccttcttttagtatatgctggtaacagcaaaaaagcgccaaacaacgcatctttacccaatagaatgacgtcgattaaaagagtaagttggtggtgagccttaaaaaaggtaaaagagagcttactcttaagttaaaaacggaattggcaaacagtaaggattgttccagcagtccttactaaaaacattactttattaattctatatagtcacaacattcacaaaaataggcttttttctcgatttttccttttgaatgtttataaagaaaaatcatatagcatttattacactgtggacatattttcttttcaattttgccccttccaatttgtggaagccttttttcagtaacacgcttaggcataatgaaagatcggtgtattaagtattaacattttctaacttgtgcaataataattattcggggttgtcgaagaacttccaaacagtagaccgatccattttccacattattctaatgaagccaagactcaaggatggggggaaatatccccccacaccccccaatgagactacccaagacagcccgtatgccgacagcgacacagttaaatatcaatgtgtattaatacatatcaacaagtatcaacacacacaattaaacaataatacacatcaatggagaacaatacacatgccaacacaagaatacacacggacaaacattacattaccagaacatgttagagcaatgattaaagagtacaataaaagaagtacatggggagacctcaacgtatcagcagtatgctctaatgcaatagcagacagaattagagctacattcccaaaattagaaaaagaactatttccagaacaatttccagaacttaacccaaaaatgacaaaaaccagctcaccaggacaaccagaaataacacaaaaaggaaaatccaaacaaggagactctaaagatagaatatgcgcttattgtggccaaccttttgaaccgaaatcgcataatcaaaaattctgtaaagatgcatgtaagtcagcaaattatagaaaaaacaaaaaaacacagagttaaacccattaaccacactctttttttgaatttatctgcatttcacggtactttttatccaagaaagcccacaaatcacgctctttaaacggtttcatggaaaaattttcccggaatgcattaattaacatcttttcaattttagggttgaaataagccccataacggtaatctggcgaccaatctgaagctttaacctcttcataagtccgatacggcacattacccggaagaagcccaggaaccacaagcagataatagatatagcaccttatacacatatgtgattttttagtagtctttaaccattcaatatatgaaagatccgcacctgttcgataatcctcaaaaagaattaagttcctttgcctccgagatccacacacacagcaatacttcaattccataatacataacaccccgaaataataagacatctgaaaaaccgtatcactttaggctttaagcatcttatccggaccttcgagcccaaaagccttgttgaacgtggcatctcaatcacccccaaacatcatggagcgtgattttaagcttgtaaatagctcttcataattaatttcggaagccttttgcaagctgttagaaggcatttcaggcgattcttcaattatacagccatcatgctcccattgatgagagtcaaccaccaggaagccggcagggggatcaggaagcccttctaagaaccatacccgaagaagactcccggcaggttcaagcttcaaaaactccggtaattcatccactttgaagggagaagcccataatttacgccattttttaggaactttgtttttgccatctcgcccttttaggttatgataaaaattatttcttgcaattaattgttgcaagtaatcggcatattcaggtttaaccgctttagcttccttcaaataagcatgtagagcctgtcttccacccgaaaaatgatcccgaagctttgttaacggtatccatccagtatcatcgttttcctgtccttcccacttcaactctccattataaacgagttttacttctcgaccttcattgattaaattaaggacttcggttctggtatctatcaattgctcaaatgtcagctttccggaggaacttacaactttatcaggatgccaccggaacaaagcaccaaagcgagattctatatggatattaccgtatctgttatccgtgagctgcccaacgtgagaataaagataagtcacctgcttgtaaatatccataaggctccgcaattcccattgccttttaccattgactttatgagaaagcctctgaataaatatatcatattcatcactcttgcatctctttttactcttcggattctttccagttatacgaatgtttccagggaaaatgaaaaaatgaaaatgaggagcaaggataaggcagtcataaatagagtttatttcgatatttagcaagtccgcgatcttgtttaaattgccttcgttgtggtcgtctcttatgtaattccaaaaagcttcggagtcgtcattaccttttaaacctgttaacttacggatagcccgagttatttccggcttaatcctgtaagggtgaaagattgtagtaccagcagttacgccccagcgttccagcctgtcctttaaattcctccggaaaagtcggattgcttccacagatactccccggctaaagtccatcgaagcaaacccccatgaaggaaactgcccagaaaacagggcataagtccacaaaattacaactaatgcaaatctatgttgcaattccgataggatgccgcaaccaggacaagaaagtgtattgcagtgagaattaagatcctttcgtgcattataattttcagatacagcataagtgaaaggacgcccacagtaagcgggtctttttccctttgctggaagaacaaagtcgccaacttttagaggacgatacttagagcatttagaaaaatcgaagtcaggacaagagtagtatttacagatagaaaccccgttctttttgttgcgaaccgggcacaaaacaccatcggtacatatacacgtcgcaaaatctctacactctggagcataggaaacggcgccaggtctagaactaaaatcagaactcagagaacatttcctgtcgaaaatttaaaccgtaaaccaaagataacaattttcataatattacagaacctatattttttgtaactcgacattacaaaaatgtaccacaaaacataaaaacattttcatagaagaaatattcacaaacaatagaaaaaaattataagaataagattaacgcctaccttattaaaagaaggtgtcgagtcttcaggttccatcaaggcaggcgttgatattctatatatgttacacataacatatataacatacgtacctcagaacattcagacctgaatgctctaaaatgaaataagctttaaactttataaattcatctgctataaatagttttagagatatagaaagcccaaggaagaaaaagaatagcaaaaataaaaaagaaaaacggcccgcgacggcaaacccggagacttataatcaaagtaagacgaaaaggaagccgacaacgatttgataataacaaatcatcttttattttggcaatattcatatgatatatgaagaaatcaacatcaagcggattagtatttttgctatttgtaaatcaatatcgggtgagacagtatatcaaaaacaaacttcatttttttcgaacaaaaaaatcactatcaaatgacattgtagtaagaaactacatcaaaactagttgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacg

SEQ ID NO: 10 - DNA, 8728 bp, Artificial
  Vector pACPur
gaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagcttgcatgcatcttctattttcatcttaattttatatattaaaggctaatttttcaatattttatcgcaaactattgtgttaattacatcaaatatataaacttttctatttaaagctaatgaaaaagtgaatatatacacatagagtaatgttatgatgtatatatcaaaaaaataggagtgattcctatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagttaattactaatttattattaatttattattagattgggcaaaatagtaaaagaaaactaaaggaaacctaatatggtttcctttttttatatatttttaaaaattgattatattatatgtaaacaattataattaatgttaatcgaattcaatttttatttaataaacggcgcgccacgcgtccttcaattgccgcgatcgttaattaaccgccacctaggcctctgcggccgcactagtaagttatagaaatgtcatccgataccgaaaaagaaaacggaataaatcagtaacaaatcaaaaaaacaaaactataacatttaatcttcgtaaaatcaatatcaatcgaccatacacaaaacatgttcaaataaaatacatgggtgtacatatgtcaaccgacattgaaaaagaaaacggaataaatcagtaacaaatcaaaaaaacaaaaccataacatttctataacattccatagagtaaaaatcaacatcgaataaccttacacaaaccatacataaaccatacataaaaatacacatatgtcaaccgttaccgtaaaaagattacttaaataattttatactcacctcttattttttgtgttttttattatccagggcaacctttcccagggagaaattcttccttgcagttgcaggcaagaaccaggactttcttccagggcatactttcccagggagaaattcttccttgcagctgcaggcaagaaccaggactttcccccaggaccaggacctcaaggtctgcatccagaggaagaccttcccagggcgccaagatcccgcagattatggaacattatttcccgggaaataattcagggcttaatgcccaactgaggaatacattttaaataagcttctcttagttcatccttttctatgtggtcgtacacgtctattgcttcgcctctcgaatctccccttaattcctgaatgaatgtcctttccatacctgaacgtcgaagccatgtagtgaaccaatgccggcagcaatgcggagtaaaatgttcttcaagtctcccctcggggttatggagtccgatagaggaagcggcagatgtgacaatatcgtacactaaatttctttgtacacggtgaccgttttgtctgcctatgaataatgctttttctttatcatgcctcgtgttaaggtacgcttccaggaaggccttagtctcaaggtcaaagaatacgatacgatttgaccttttagctgtcttcttgagtctcaccgtacacttttccagatctatgtcagctcgatcaagagtaattaattcatttctcctgatgccagttttagctaaaaagattatcaatgcttgatacatggggtaacgtggtactttgacaagctttgccatgtcttcaatggtaatcaattgccttgtttccggagtatggtattttttgtagcttcttagatagcgttttctgaacgtgggaatgttgtttctctcgattatatgcttgaattcaaggaagtcaaaaaaggtactcagtgaagcaaagtaattgttacaggttgcgagtgtgtactgcctttcatctcttatgtgaacaaggaacccttcaagctccggaaaccctgcattcactccacacatttgaaggaaaaacttagcatgacttacgtatccttctattgttcttttagagtaaccccttccgatacaatcagaagtgaataaacttataagtctttcattatcaagcggttcatagataccagttagctgaatagctgccatttcacccataaatcaataaaagaagtatgtaaataaatactttgtgcatagatgtcatttaaacacaaagtataagtacatctaccttcttttagtatatgctggtaacagcaaaaaagcgccaaacaacgcatctttacccaatagaatgacgtcgattaaaagagtaagttggtggtgagccttaaaaaaggtaaaagagagcttactcttaagttaaaaacggaattggcaaacagtaaggattgttccagcagtccttactaaaaacattactttattaattctatatagtcacaacattcacaaaaataggcttttttctcgatttttccttttgaatgtttataaagaaaaatcatatagcatttattacactgtggacatattttcttttcaattttgccccttccaatttgtggaagccttttttcagtaacacgcttaggcataatgaaagatcggtgtattaagtattaacattttctaacttgtgcaataataattattcggggttgtcgaagaacttccaaacagtagaccgatccattttccacattattctaatgaagccaagactcaaggatggggggaaatatccccccacaccccccaatgagactacccaagacagcccgtatgccgacagcgacacagttaaatatcaatgtgtattaatacatatcaacaagtatcaacacacacaattaaacaataatacacatcaatggagaacaatacacatgccaacacaagaatacacacggacaaacattacattaccagaacatgttagagcaatgattaaagagtacaataaaagaagtacatggggagacctcaacgtatcagcagtatgctctaatgcaatagcagacagaattagagctacattcccaaaattagaaaaagaactatttccagaacaatttccagaacttaacccaaaaatgacaaaaaccagctcaccaggacaaccagaaataacacaaaaaggaaaatccaaacaaggagactctaaagatagaatatgcgcttattgtggccaaccttttgaaccgaaatcgcataatcaaaaattctgtaaagatgcatgtaagtcagcaaattatagaaaaaacaaaaaaacacagagttaaacccattaaccacactctttttttgaatttatctgcatttcacggtactttttatccaagaaagcccacaaatcacgctctttaaacggtttcatggaaaaattttcccggaatgcattaattaacatcttttcaattttagggttgaaataagccccataacggtaatctggcgaccaatctgaagctttaacctcttcataagtccgatacggcacattacccggaagaagcccaggaaccacaagcagataatagatatagcaccttatacacatatgtgattttttagtagtctttaaccattcaatatatgaaagatccgcacctgttcgataatcctcaaaaagaattaagttcctttgcctccgagatccacacacacagcaatacttcaattccataatacataacaccccgaaataataagacatctgaaaaaccgtatcactttaggctttaagcatcttatccggaccttcgagcccaaaagccttgttgaacgtggcatctcaatcacccccaaacatcatggagcgtgattttaagcttgtaaatagctcttcataattaatttcggaagccttttgcaagctgttagaaggcatttcaggcgattcttcaattatacagccatcatgctcccattgatgagagtcaaccaccaggaagccggcagggggatcaggaagcccttctaagaaccatacccgaagaagactcccggcaggttcaagcttcaaaaactccggtaattcatccactttgaagggagaagcccataatttacgccattttttaggaactttgtttttgccatctcgcccttttaggttatgataaaaattatttcttgcaattaattgttgcaagtaatcggcatattcaggtttaaccgctttagcttccttcaaataagcatgtagagcctgtcttccacccgaaaaatgatcccgaagctttgttaacggtatccatccagtatcatcgttttcctgtccttcccacttcaactctccattataaacgagttttacttctcgaccttcattgattaaattaaggacttcggttctggtatctatcaattgctcaaatgtcagctttccggaggaacttacaactttatcaggatgccaccggaacaaagcaccaaagcgagattctatatggatattaccgtatctgttatccgtgagctgcccaacgtgagaataaagataagtcacctgcttgtaaatatccataaggctccgcaattcccattgccttttaccattgactttatgagaaagcctctgaataaatatatcatattcatcactcttgcatctctttttactcttcggattctttccagttatacgaatgtttccagggaaaatgaaaaaatgaaaatgaggagcaaggataaggcagtcataaatagagtttatttcgatatttagcaagtccgcgatcttgtttaaattgccttcgttgtggtcgtctcttatgtaattccaaaaagcttcggagtcgtcattaccttttaaacctgttaacttacggatagcccgagttatttccggcttaatcctgtaagggtgaaagattgtagtaccagcagttacgccccagcgttccagcctgtcctttaaattcctccggaaaagtcggattgcttccacagatactccccggctaaagtccatcgaagcaaacccccatgaaggaaactgcccagaaaacagggcataagtccacaaaattacaactaatgcaaatctatgttgcaattccgataggatgccgcaaccaggacaagaaagtgtattgcagtgagaattaagatcctttcgtgcattataattttcagatacagcataagtgaaaggacgcccacagtaagcgggtctttttccctttgctggaagaacaaagtcgccaacttttagaggacgatacttagagcatttagaaaaatcgaagtcaggacaagagtagtatttacagatagaaaccccgttctttttgttgcgaaccgggcacaaaacaccatcggtacatatacacgtcgcaaaatctctacactctggagcataggaaacggcgccaggtctagaactaaaatcagaactcagagaacatttcctgtcgaaaatttaaaccgtaaaccaaagataacaattttcataatattacagaacctatattttttgtaactcgacattacaaaaatgtaccacaaaacataaaaacattttcatagaagaaatattcacaaacaatagaaaaaaattataagaataagattaacgcctaccttattaaaagaaggtgtcgagtcttcaggttccatcaaggcaggcgttgatattctatatatgttacacataacatatataacatacgtacctcagaacattcagacctgaatgctctaaaatgaaataagctttaaactttataaattcatctgctataaatagttttagagatatagaaagcccaaggaagaaaaagaatagcaaaaataaaaaagaaaaacggcccgcgacggcaaacccggagacttataatcaaagtaagacgaaaaggaagccgacaacgatttgataataacaaatcatcttttattttggcaatattcatatgatatatgaagaaatcaacatcaagcggattagtatttttgctatttgtaaatcaatatcgggtgagacagtatatcaaaaacaaacttcatttttttcgaacaaaaaaatcactatcaaatgacattgtagtaagaaactacatcaaaactagttgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacg

SEQ ID NO: 11 - DNA, 3008 bp, Artificial
  DNA fragment encoding IDI-FnS
ttaattaaatgatttttaaaaaaatacatgaattcatctagcggagaacacaaaagatttaagtaccttctaaacgaatgagatttcattgggaaagtggacacttaagaacgacgcggtacttgatttattgagtgcaaaagcactcgattaggtgaccagtcccaaagtgattttaataaattaaggaggaaattaaaatgactgccgacaacaatagtatgccccatggtgcagtatctagttacgccaaattagtgcaaaaccaaacacctgaagacattttggaagagtttcctgaaattattccattacaacaaagacctaatacccgatctagtgagacgtcaaatgacgaaagcggagaaacatgtttttctggtcatgatgaggagcaaattaagttaatgaatgaaaattgtattgttttggattgggacgataatgctattggtgccggtaccaagaaagtttgtcatttaatggaaaatattgaaaagggtttactacatcgtgcattctccgtctttattttcaatgaacaaggtgaattacttttacaacaaagagccactgaaaaaataactttccctgatctttggactaacacatgctgctctcatccactatgtattgatgacgaattaggtttgaagggtaagctagacgataagattaagggcgctattactgcggcggtgagaaaactagatcatgaattaggtattccagaagatgaaactaagacaaggggtaagtttcactttttaaacagaatccattacatggcaccaagcaatgaaccatggggtgaacatgaaattgattacatcctattttataagatcaacgctaaagaaaacttgactgtcaacccaaacgtcaatgaagttagagacttcaaatgggtttcaccaaatgatttgaaaactatgtttgctgacccaagttacaagtttacgccttggtttaagattatttgcgagaattacttattcaactggtgggagcaattagatgacctttctgaagtggaaaatgacaggcaaattcatagaatgctataaacaaattagttaaaaggactaaagatgtcgactcttcctatttctagtgtttcattctcttcatctacgtcacctttggttgttgacgataaagttagcacgaaaccagatgttatccgccataccatgaatttcaatgctagcatatggggagatcaatttcttacatatgatgagccagaagatttggttatgaagaaacaacttgttgaagagctcaaagaggaagtcaagaaagagctaataacgatcaaaggttcaaatgaaccgatgcagcatgtcaagttgatagaacttattgatgcagtccaacggcttggcatagcctatcattttgaagaggagattgaagaagccttgcaacatatccatgttacatatggcgagcagtgggtcgataaagaaaacctacaaagcatttctctttggttccgactcctacgacaacaaggcttcaacgtctcatcaggagtattcaaggactttatggacgagaagggaaaatttaaggaatccttatgtaatgatgctcagggaattcttgctctgtatgaagcggcatttatgagggtggaagatgaaacaatactagataatgcgctcgagttcactaaagttcaccttgacatcatagccaaggatccttcttgtgactcttccctaagaacccaaatacaccaagcgttaaagcagccacttaggagaaggctggcaaggattgaggcgttgcattacatgcctatctaccaacaagaaacatcccacgatgaggtcttactgaagcttgcaaaattagattttagcgtgcttcagtcgatgcacaaaaaggaacttagccacatttgcaaatggtggaaggatttggacctccaaaataagcttccttatgttcgagacagggtggtggaaggctatttttggatactatccatctattacgagcctcaacatgctcgaacaagaatgttcctaatgaaaacatgcatgtggttagtcgttttagatgatacatttgataattatggtacttatgaagaactcgaaatctttacacaagctgttgaaagatggtcaataagctgcctggatatgcttccagaatacatgaaactaatatatcaagagcttgtgaatcttcacgtggaaatggaggaatcacttgaaaaggagggaaaaacatatcaaattcactatgtcaaggagatggcaaaagagttggttcgcaattacttggtagaagccagatggctaaaagaggggtacatgccaactcttgaggagtacatgtctgtgtcaatggtgactggtacctatggcttgatgatagcgagatcttatgtcggcaggggtgatatcgtcaccgaggatacctttaaatgggtgtcctcgtatcctcctattataaaagcttcatgtgtgattgtaagacttatggatgatattgtcagccacaaggaggaacaagagagaggccatgttgcttcaagcatcgaatgctattctaaggaaagtggtgcatcagaggaggaagcgtgtgaatatatctcaagaaaagttgaagatgcatggaaagttataaaccgagagtcgctcaggcctacagctgtcccatttcctctacttatgcctgcaatcaaccttgcacgtatgtgtgaagtcctatatagcgtcaacgatggcttcactcatgctgagggagacatgaaaagttacatgaaatcgttcttcgttcaccctatggttgtctaaactcaataaattaaacaacttaaaacagagcaggccttcgggcctgctttttctttctttcccgagtcccgcctcgggagggcggtgtcctttccgccccgaattgcgcatcgggagcacgcggtccttttcgctcgctttgctctctcaagaggactaatttacaaaattacatattgagagcgcggccgc

SEQ ID NO: 12 - DNA, 4083 bp, Artificial
  DNA fragment encoding IDI-FnS-FPS
ttaattaaatgatttttaaaaaaatacatgaattcatctagcggagaacacaaaagatttaagtaccttctaaacgaatgagatttcattgggaaagtggacacttaagaacgacgcggtacttgatttattgagtgcaaaagcactcgattaggtgaccagtcccaaagtgattttaataaattaaggaggaaattaaaatgactgccgacaacaatagtatgccccatggtgcagtatctagttacgccaaattagtgcaaaaccaaacacctgaagacattttggaagagtttcctgaaattattccattacaacaaagacctaatacccgatctagtgagacgtcaaatgacgaaagcggagaaacatgtttttctggtcatgatgaggagcaaattaagttaatgaatgaaaattgtattgttttggattgggacgataatgctattggtgccggtaccaagaaagtttgtcatttaatggaaaatattgaaaagggtttactacatcgtgcattctccgtctttattttcaatgaacaaggtgaattacttttacaacaaagagccactgaaaaaataactttccctgatctttggactaacacatgctgctctcatccactatgtattgatgacgaattaggtttgaagggtaagctagacgataagattaagggcgctattactgcggcggtgagaaaactagatcatgaattaggtattccagaagatgaaactaagacaaggggtaagtttcactttttaaacagaatccattacatggcaccaagcaatgaaccatggggtgaacatgaaattgattacatcctattttataagatcaacgctaaagaaaacttgactgtcaacccaaacgtcaatgaagttagagacttcaaatgggtttcaccaaatgatttgaaaactatgtttgctgacccaagttacaagtttacgccttggtttaagattatttgcgagaattacttattcaactggtgggagcaattagatgacctttctgaagtggaaaatgacaggcaaattcatagaatgctataaacaaattagttaaaaggactaaagatgtcgactcttcctatttctagtgtttcattctcttcatctacgtcacctttggttgttgacgataaagttagcacgaaaccagatgttatccgccataccatgaatttcaatgctagcatatggggagatcaatttcttacatatgatgagccagaagatttggttatgaagaaacaacttgttgaagagctcaaagaggaagtcaagaaagagctaataacgatcaaaggttcaaatgaaccgatgcagcatgtcaagttgatagaacttattgatgcagtccaacggcttggcatagcctatcattttgaagaggagattgaagaagccttgcaacatatccatgttacatatggcgagcagtgggtcgataaagaaaacctacaaagcatttctctttggttccgactcctacgacaacaaggcttcaacgtctcatcaggagtattcaaggactttatggacgagaagggaaaatttaaggaatccttatgtaatgatgctcagggaattcttgctctgtatgaagcggcatttatgagggtggaagatgaaacaatactagataatgcgctcgagttcactaaagttcaccttgacatcatagccaaggatccttcttgtgactcttccctaagaacccaaatacaccaagcgttaaagcagccacttaggagaaggctggcaaggattgaggcgttgcattacatgcctatctaccaacaagaaacatcccacgatgaggtcttactgaagcttgcaaaattagattttagcgtgcttcagtcgatgcacaaaaaggaacttagccacatttgcaaatggtggaaggatttggacctccaaaataagcttccttatgttcgagacagggtggtggaaggctatttttggatactatccatctattacgagcctcaacatgctcgaacaagaatgttcctaatgaaaacatgcatgtggttagtcgttttagatgatacatttgataattatggtacttatgaagaactcgaaatctttacacaagctgttgaaagatggtcaataagctgcctggatatgcttccagaatacatgaaactaatatatcaagagcttgtgaatcttcacgtggaaatggaggaatcacttgaaaaggagggaaaaacatatcaaattcactatgtcaaggagatggcaaaagagttggttcgcaattacttggtagaagccagatggctaaaagaggggtacatgccaactcttgaggagtacatgtctgtgtcaatggtgactggtacctatggcttgatgatagcgagatcttatgtcggcaggggtgatatcgtcaccgaggatacctttaaatgggtgtcctcgtatcctcctattataaaagcttcatgtgtgattgtaagacttatggatgatattgtcagccacaaggaggaacaagagagaggccatgttgcttcaagcatcgaatgctattctaaggaaagtggtgcatcagaggaggaagcgtgtgaatatatctcaagaaaagttgaagatgcatggaaagttataaaccgagagtcgctcaggcctacagctgtcccatttcctctacttatgcctgcaatcaaccttgcacgtatgtgtgaagtcctatatagcgtcaacgatggcttcactcatgctgagggagacatgaaaagttacatgaaatcgttcttcgttcaccctatggttgtctaaaaaggaggagatcataatggcttcagaaaaagaaattaggagagagagattcttgaacgttttccctaaattagtagaggaattgaacgcatcgcttttggcttacggtatgcctaaggaagcatgtgactggtatgcccactcattgaactacaacactccaggcggtaagctaaatagaggtttgtccgttgtggacacgtatgctattctctccaacaagaccgttgaacaattggggcaagaagaatacgaaaaggttgccattctaggttggtgcattgagttgttgcaggcttacttcttggtcgccgatgatatgatggacaagtccattaccagaagaggccaaccatgttggtacaaggttcctgaagttggggaaattgccatcaatgacgcattcatgttagaggctgctatctacaagcttttgaaatctcacttcagaaacgaaaaatactacatagatatcaccgaattgttccatgaggtcaccttccaaaccgaattgggccaattgatggacttaatcactgcacctgaagacaaagtcgacttgagtaagttctccctaaagaagcactccttcatagttactttcaagactgcttactattctttctacttgcctgtcgcattggccatgtacgttgccggtatcacggatgaaaaggatttgaaacaagccagagatgtcttgattccattgggtgaatacttccaaattcaagatgactacttagactgcttcggtaccccagaacagatcggtaagatcggtacagatatccaagataacaaatgttcttgggtaatcaacaaggcattggaacttgcttccgcagaacaaagaaagactttagacgaaaattacggtaagaaggactcagtcgcagaagccaaatgcaaaaagattttcaatgacttgaaaattgaacagctataccacgaatatgaagagtctattgccaaggatttgaaggccaaaatttctcaggtcgatgagtctcgtggcttcaaagctgatgtcttaactgcgttcttgaacaaagtttacaagagaagcaaatagactcaataaattaaacaacttaaaacagagcaggccttcgggcctgctttttctttctttcccgagtcccgcctcgggagggcggtgtcctttccgccccgaattgcgcatcgggagcacgcggtccttttcgctcgctttgctctctcaagaggactaatttacaaaattacatattgagagcgcggccgc

SEQ ID NO: 13 - DNA, 11703 bp, Artificial
  Vector pACPmcr-IDI-FnS
gaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagcttgcatgcatcttctattttcatcttaattttatatattaaaggctaatttttcaatattttatcgcaaactattgtgttaattacatcaaatatataaacttttctatttaaagctaatgaaaaagtgaatatatacacatagagtaatgttatgatgtatatatcaaaaaaataggagtgattcctatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagttaattactaatttattattaatttattattagattgggcaaaatagtaaaagaaaactaaaggaaacctaatatggtttcctttttttatatatttttaaaaattgattatattatatgtaaacaattataattaatgttaatcgaattcaatttttatttaataaacggcgcgccacgcgtccttcaattgccgcgatcgttaattaaatgatttttaaaaaaatacatgaattcatctagcggagaacacaaaagatttaagtaccttctaaacgaatgagatttcattgggaaagtggacacttaagaacgacgcggtacttgatttattgagtgcaaaagcactcgattaggtgaccagtcccaaagtgattttaataaattaaggaggaaattaaaatgactgccgacaacaatagtatgccccatggtgcagtatctagttacgccaaattagtgcaaaaccaaacacctgaagacattttggaagagtttcctgaaattattccattacaacaaagacctaatacccgatctagtgagacgtcaaatgacgaaagcggagaaacatgtttttctggtcatgatgaggagcaaattaagttaatgaatgaaaattgtattgttttggattgggacgataatgctattggtgccggtaccaagaaagtttgtcatttaatggaaaatattgaaaagggtttactacatcgtgcattctccgtctttattttcaatgaacaaggtgaattacttttacaacaaagagccactgaaaaaataactttccctgatctttggactaacacatgctgctctcatccactatgtattgatgacgaattaggtttgaagggtaagctagacgataagattaagggcgctattactgcggcggtgagaaaactagatcatgaattaggtattccagaagatgaaactaagacaaggggtaagtttcactttttaaacagaatccattacatggcaccaagcaatgaaccatggggtgaacatgaaattgattacatcctattttataagatcaacgctaaagaaaacttgactgtcaacccaaacgtcaatgaagttagagacttcaaatgggtttcaccaaatgatttgaaaactatgtttgctgacccaagttacaagtttacgccttggtttaagattatttgcgagaattacttattcaactggtgggagcaattagatgacctttctgaagtggaaaatgacaggcaaattcatagaatgctataaacaaattagttaaaaggactaaagatgtcgactcttcctatttctagtgtttcattctcttcatctacgtcacctttggttgttgacgataaagttagcacgaaaccagatgttatccgccataccatgaatttcaatgctagcatatggggagatcaatttcttacatatgatgagccagaagatttggttatgaagaaacaacttgttgaagagctcaaagaggaagtcaagaaagagctaataacgatcaaaggttcaaatgaaccgatgcagcatgtcaagttgatagaacttattgatgcagtccaacggcttggcatagcctatcattttgaagaggagattgaagaagccttgcaacatatccatgttacatatggcgagcagtgggtcgataaagaaaacctacaaagcatttctctttggttccgactcctacgacaacaaggcttcaacgtctcatcaggagtattcaaggactttatggacgagaagggaaaatttaaggaatccttatgtaatgatgctcagggaattcttgctctgtatgaagcggcatttatgagggtggaagatgaaacaatactagataatgcgctcgagttcactaaagttcaccttgacatcatagccaaggatccttcttgtgactcttccctaagaacccaaatacaccaagcgttaaagcagccacttaggagaaggctggcaaggattgaggcgttgcattacatgcctatctaccaacaagaaacatcccacgatgaggtcttactgaagcttgcaaaattagattttagcgtgcttcagtcgatgcacaaaaaggaacttagccacatttgcaaatggtggaaggatttggacctccaaaataagcttccttatgttcgagacagggtggtggaaggctatttttggatactatccatctattacgagcctcaacatgctcgaacaagaatgttcctaatgaaaacatgcatgtggttagtcgttttagatgatacatttgataattatggtacttatgaagaactcgaaatctttacacaagctgttgaaagatggtcaataagctgcctggatatgcttccagaatacatgaaactaatatatcaagagcttgtgaatcttcacgtggaaatggaggaatcacttgaaaaggagggaaaaacatatcaaattcactatgtcaaggagatggcaaaagagttggttcgcaattacttggtagaagccagatggctaaaagaggggtacatgccaactcttgaggagtacatgtctgtgtcaatggtgactggtacctatggcttgatgatagcgagatcttatgtcggcaggggtgatatcgtcaccgaggatacctttaaatgggtgtcctcgtatcctcctattataaaagcttcatgtgtgattgtaagacttatggatgatattgtcagccacaaggaggaacaagagagaggccatgttgcttcaagcatcgaatgctattctaaggaaagtggtgcatcagaggaggaagcgtgtgaatatatctcaagaaaagttgaagatgcatggaaagttataaaccgagagtcgctcaggcctacagctgtcccatttcctctacttatgcctgcaatcaaccttgcacgtatgtgtgaagtcctatatagcgtcaacgatggcttcactcatgctgagggagacatgaaaagttacatgaaatcgttcttcgttcaccctatggttgtctaaactcaataaattaaacaacttaaaacagagcaggccttcgggcctgctttttctttctttcccgagtcccgcctcgggagggcggtgtcctttccgccccgaattgcgcatcgggagcacgcggtccttttcgctcgctttgctctctcaagaggactaatttacaaaattacatattgagagcgcggccgcactagtaagttatagaaatgtcatccgataccgaaaaagaaaacggaataaatcagtaacaaatcaaaaaaacaaaactataacatttaatcttcgtaaaatcaatatcaatcgaccatacacaaaacatgttcaaataaaatacatgggtgtacatatgtcaaccgacattgaaaaagaaaacggaataaatcagtaacaaatcaaaaaaacaaaaccataacatttctataacattccatagagtaaaaatcaacatcgaataaccttacacaaaccatacataaaccatacataaaaatacacatatgtcaaccgttaccgtaaaaagattacttaaataattttatactcacctcttattttttgtgttttttattatccagggcaacctttcccagggagaaattcttccttgcagttgcaggcaagaaccaggactttcttccagggcatactttcccagggagaaattcttccttgcagctgcaggcaagaaccaggactttcccccaggaccaggacctcaaggtctgcatccagaggaagaccttcccagggcgccaagatcccgcagattatggaacattatttcccgggaaataattcagggcttaatgcccaactgaggaatacattttaaataagcttctcttagttcatccttttctatgtggtcgtacacgtctattgcttcgcctctcgaatctccccttaattcctgaatgaatgtcctttccatacctgaacgtcgaagccatgtagtgaaccaatgccggcagcaatgcggagtaaaatgttcttcaagtctcccctcggggttatggagtccgatagaggaagcggcagatgtgacaatatcgtacactaaatttctttgtacacggtgaccgttttgtctgcctatgaataatgctttttctttatcatgcctcgtgttaaggtacgcttccaggaaggccttagtctcaaggtcaaagaatacgatacgatttgaccttttagctgtcttcttgagtctcaccgtacacttttccagatctatgtcagctcgatcaagagtaattaattcatttctcctgatgccagttttagctaaaaagattatcaatgcttgatacatggggtaacgtggtactttgacaagctttgccatgtcttcaatggtaatcaattgccttgtttccggagtatggtattttttgtagcttcttagatagcgttttctgaacgtgggaatgttgtttctctcgattatatgcttgaattcaaggaagtcaaaaaaggtactcagtgaagcaaagtaattgttacaggttgcgagtgtgtactgcctttcatctcttatgtgaacaaggaacccttcaagctccggaaaccctgcattcactccacacatttgaaggaaaaacttagcatgacttacgtatccttctattgttcttttagagtaaccccttccgatacaatcagaagtgaataaacttataagtctttcattatcaagcggttcatagataccagttagctgaatagctgccatttcacccataaatcaataaaagaagtatgtaaataaatactttgtgcatagatgtcatttaaacacaaagtataagtacatctaccttcttttagtatatgctggtaacagcaaaaaagcgccaaacaacgcatctttacccaatagaatgacgtcgattaaaagagtaagttggtggtgagccttaaaaaaggtaaaagagagcttactcttaagttaaaaacggaattggcaaacagtaaggattgttccagcagtccttactaaaaacattactttattaattctatatagtcacaacattcacaaaaataggcttttttctcgatttttccttttgaatgtttataaagaaaaatcatatagcatttattacactgtggacatattttcttttcaattttgccccttccaatttgtggaagccttttttcagtaacacgcttaggcataatgaaagatcggtgtattaagtattaacattttctaacttgtgcaataataattattcggggttgtcgaagaacttccaaacagtagaccgatccattttccacattattctaatgaagccaagactcaaggatggggggaaatatccccccacaccccccaatgagactacccaagacagcccgtatgccgacagcgacacagttaaatatcaatgtgtattaatacatatcaacaagtatcaacacacacaattaaacaataatacacatcaatggagaacaatacacatgccaacacaagaatacacacggacaaacattacattaccagaacatgttagagcaatgattaaagagtacaataaaagaagtacatggggagacctcaacgtatcagcagtatgctctaatgcaatagcagacagaattagagctacattcccaaaattagaaaaagaactatttccagaacaatttccagaacttaacccaaaaatgacaaaaaccagctcaccaggacaaccagaaataacacaaaaaggaaaatccaaacaaggagactctaaagatagaatatgcgcttattgtggccaaccttttgaaccgaaatcgcataatcaaaaattctgtaaagatgcatgtaagtcagcaaattatagaaaaaacaaaaaaacacagagttaaacccattaaccacactctttttttgaatttatctgcatttcacggtactttttatccaagaaagcccacaaatcacgctctttaaacggtttcatggaaaaattttcccggaatgcattaattaacatcttttcaattttagggttgaaataagccccataacggtaatctggcgaccaatctgaagctttaacctcttcataagtccgatacggcacattacccggaagaagcccaggaaccacaagcagataatagatatagcaccttatacacatatgtgattttttagtagtctttaaccattcaatatatgaaagatccgcacctgttcgataatcctcaaaaagaattaagttcctttgcctccgagatccacacacacagcaatacttcaattccataatacataacaccccgaaataataagacatctgaaaaaccgtatcactttaggctttaagcatcttatccggaccttcgagcccaaaagccttgttgaacgtggcatctcaatcacccccaaacatcatggagcgtgattttaagcttgtaaatagctcttcataattaatttcggaagccttttgcaagctgttagaaggcatttcaggcgattcttcaattatacagccatcatgctcccattgatgagagtcaaccaccaggaagccggcagggggatcaggaagcccttctaagaaccatacccgaagaagactcccggcaggttcaagcttcaaaaactccggtaattcatccactttgaagggagaagcccataatttacgccattttttaggaactttgtttttgccatctcgcccttttaggttatgataaaaattatttcttgcaattaattgttgcaagtaatcggcatattcaggtttaaccgctttagcttccttcaaataagcatgtagagcctgtcttccacccgaaaaatgatcccgaagctttgttaacggtatccatccagtatcatcgttttcctgtccttcccacttcaactctccattataaacgagttttacttctcgaccttcattgattaaattaaggacttcggttctggtatctatcaattgctcaaatgtcagctttccggaggaacttacaactttatcaggatgccaccggaacaaagcaccaaagcgagattctatatggatattaccgtatctgttatccgtgagctgcccaacgtgagaataaagataagtcacctgcttgtaaatatccataaggctccgcaattcccattgccttttaccattgactttatgagaaagcctctgaataaatatatcatattcatcactcttgcatctctttttactcttcggattctttccagttatacgaatgtttccagggaaaatgaaaaaatgaaaatgaggagcaaggataaggcagtcataaatagagtttatttcgatatttagcaagtccgcgatcttgtttaaattgccttcgttgtggtcgtctcttatgtaattccaaaaagcttcggagtcgtcattaccttttaaacctgttaacttacggatagcccgagttatttccggcttaatcctgtaagggtgaaagattgtagtaccagcagttacgccccagcgttccagcctgtcctttaaattcctccggaaaagtcggattgcttccacagatactccccggctaaagtccatcgaagcaaacccccatgaaggaaactgcccagaaaacagggcataagtccacaaaattacaactaatgcaaatctatgttgcaattccgataggatgccgcaaccaggacaagaaagtgtattgcagtgagaattaagatcctttcgtgcattataattttcagatacagcataagtgaaaggacgcccacagtaagcgggtctttttccctttgctggaagaacaaagtcgccaacttttagaggacgatacttagagcatttagaaaaatcgaagtcaggacaagagtagtatttacagatagaaaccccgttctttttgttgcgaaccgggcacaaaacaccatcggtacatatacacgtcgcaaaatctctacactctggagcataggaaacggcgccaggtctagaactaaaatcagaactcagagaacatttcctgtcgaaaatttaaaccgtaaaccaaagataacaattttcataatattacagaacctatattttttgtaactcgacattacaaaaatgtaccacaaaacataaaaacattttcatagaagaaatattcacaaacaatagaaaaaaattataagaataagattaacgcctaccttattaaaagaaggtgtcgagtcttcaggttccatcaaggcaggcgttgatattctatatatgttacacataacatatataacatacgtacctcagaacattcagacctgaatgctctaaaatgaaataagctttaaactttataaattcatctgctataaatagttttagagatatagaaagcccaaggaagaaaaagaatagcaaaaataaaaaagaaaaacggcccgcgacggcaaacccggagacttataatcaaagtaagacgaaaaggaagccgacaacgatttgataataacaaatcatcttttattttggcaatattcatatgatatatgaagaaatcaacatcaagcggattagtatttttgctatttgtaaatcaatatcgggtgagacagtatatcaaaaacaaacttcatttttttcgaacaaaaaaatcactatcaaatgacattgtagtaagaaactacatcaaaactagttgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacg

SEQ ID NO: 14 - DNA, 12778 bp, Artificial
  pACPmcr-IDI-FnS-FPS
gaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagcttgcatgcatcttctattttcatcttaattttatatattaaaggctaatttttcaatattttatcgcaaactattgtgttaattacatcaaatatataaacttttctatttaaagctaatgaaaaagtgaatatatacacatagagtaatgttatgatgtatatatcaaaaaaataggagtgattcctatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagttaattactaatttattattaatttattattagattgggcaaaatagtaaaagaaaactaaaggaaacctaatatggtttcctttttttatatatttttaaaaattgattatattatatgtaaacaattataattaatgttaatcgaattcaatttttatttaataaacggcgcgccacgcgtccttcaattgccgcgatcgttaattaaatgatttttaaaaaaatacatgaattcatctagcggagaacacaaaagatttaagtaccttctaaacgaatgagatttcattgggaaagtggacacttaagaacgacgcggtacttgatttattgagtgcaaaagcactcgattaggtgaccagtcccaaagtgattttaataaattaaggaggaaattaaaatgactgccgacaacaatagtatgccccatggtgcagtatctagttacgccaaattagtgcaaaaccaaacacctgaagacattttggaagagtttcctgaaattattccattacaacaaagacctaatacccgatctagtgagacgtcaaatgacgaaagcggagaaacatgtttttctggtcatgatgaggagcaaattaagttaatgaatgaaaattgtattgttttggattgggacgataatgctattggtgccggtaccaagaaagtttgtcatttaatggaaaatattgaaaagggtttactacatcgtgcattctccgtctttattttcaatgaacaaggtgaattacttttacaacaaagagccactgaaaaaataactttccctgatctttggactaacacatgctgctctcatccactatgtattgatgacgaattaggtttgaagggtaagctagacgataagattaagggcgctattactgcggcggtgagaaaactagatcatgaattaggtattccagaagatgaaactaagacaaggggtaagtttcactttttaaacagaatccattacatggcaccaagcaatgaaccatggggtgaacatgaaattgattacatcctattttataagatcaacgctaaagaaaacttgactgtcaacccaaacgtcaatgaagttagagacttcaaatgggtttcaccaaatgatttgaaaactatgtttgctgacccaagttacaagtttacgccttggtttaagattatttgcgagaattacttattcaactggtgggagcaattagatgacctttctgaagtggaaaatgacaggcaaattcatagaatgctataaacaaattagttaaaaggactaaagatgtcgactcttcctatttctagtgtttcattctcttcatctacgtcacctttggttgttgacgataaagttagcacgaaaccagatgttatccgccataccatgaatttcaatgctagcatatggggagatcaatttcttacatatgatgagccagaagatttggttatgaagaaacaacttgttgaagagctcaaagaggaagtcaagaaagagctaataacgatcaaaggttcaaatgaaccgatgcagcatgtcaagttgatagaacttattgatgcagtccaacggcttggcatagcctatcattttgaagaggagattgaagaagccttgcaacatatccatgttacatatggcgagcagtgggtcgataaagaaaacctacaaagcatttctctttggttccgactcctacgacaacaaggcttcaacgtctcatcaggagtattcaaggactttatggacgagaagggaaaatttaaggaatccttatgtaatgatgctcagggaattcttgctctgtatgaagcggcatttatgagggtggaagatgaaacaatactagataatgcgctcgagttcactaaagttcaccttgacatcatagccaaggatccttcttgtgactcttccctaagaacccaaatacaccaagcgttaaagcagccacttaggagaaggctggcaaggattgaggcgttgcattacatgcctatctaccaacaagaaacatcccacgatgaggtcttactgaagcttgcaaaattagattttagcgtgcttcagtcgatgcacaaaaaggaacttagccacatttgcaaatggtggaaggatttggacctccaaaataagcttccttatgttcgagacagggtggtggaaggctatttttggatactatccatctattacgagcctcaacatgctcgaacaagaatgttcctaatgaaaacatgcatgtggttagtcgttttagatgatacatttgataattatggtacttatgaagaactcgaaatctttacacaagctgttgaaagatggtcaataagctgcctggatatgcttccagaatacatgaaactaatatatcaagagcttgtgaatcttcacgtggaaatggaggaatcacttgaaaaggagggaaaaacatatcaaattcactatgtcaaggagatggcaaaagagttggttcgcaattacttggtagaagccagatggctaaaagaggggtacatgccaactcttgaggagtacatgtctgtgtcaatggtgactggtacctatggcttgatgatagcgagatcttatgtcggcaggggtgatatcgtcaccgaggatacctttaaatgggtgtcctcgtatcctcctattataaaagcttcatgtgtgattgtaagacttatggatgatattgtcagccacaaggaggaacaagagagaggccatgttgcttcaagcatcgaatgctattctaaggaaagtggtgcatcagaggaggaagcgtgtgaatatatctcaagaaaagttgaagatgcatggaaagttataaaccgagagtcgctcaggcctacagctgtcccatttcctctacttatgcctgcaatcaaccttgcacgtatgtgtgaagtcctatatagcgtcaacgatggcttcactcatgctgagggagacatgaaaagttacatgaaatcgttcttcgttcaccctatggttgtctaaaaaggaggagatcataatggcttcagaaaaagaaattaggagagagagattcttgaacgttttccctaaattagtagaggaattgaacgcatcgcttttggcttacggtatgcctaaggaagcatgtgactggtatgcccactcattgaactacaacactccaggcggtaagctaaatagaggtttgtccgttgtggacacgtatgctattctctccaacaagaccgttgaacaattggggcaagaagaatacgaaaaggttgccattctaggttggtgcattgagttgttgcaggcttacttcttggtcgccgatgatatgatggacaagtccattaccagaagaggccaaccatgttggtacaaggttcctgaagttggggaaattgccatcaatgacgcattcatgttagaggctgctatctacaagcttttgaaatctcacttcagaaacgaaaaatactacatagatatcaccgaattgttccatgaggtcaccttccaaaccgaattgggccaattgatggacttaatcactgcacctgaagacaaagtcgacttgagtaagttctccctaaagaagcactccttcatagttactttcaagactgcttactattctttctacttgcctgtcgcattggccatgtacgttgccggtatcacggatgaaaaggatttgaaacaagccagagatgtcttgattccattgggtgaatacttccaaattcaagatgactacttagactgcttcggtaccccagaacagatcggtaagatcggtacagatatccaagataacaaatgttcttgggtaatcaacaaggcattggaacttgcttccgcagaacaaagaaagactttagacgaaaattacggtaagaaggactcagtcgcagaagccaaatgcaaaaagattttcaatgacttgaaaattgaacagctataccacgaatatgaagagtctattgccaaggatttgaaggccaaaatttctcaggtcgatgagtctcgtggcttcaaagctgatgtcttaactgcgttcttgaacaaagtttacaagagaagcaaatagactcaataaattaaacaacttaaaacagagcaggccttcgggcctgctttttctttctttcccgagtcccgcctcgggagggcggtgtcctttccgccccgaattgcgcatcgggagcacgcggtccttttcgctcgctttgctctctcaagaggactaatttacaaaattacatattgagagcgcggccgcactagtaagttatagaaatgtcatccgataccgaaaaagaaaacggaataaatcagtaacaaatcaaaaaaacaaaactataacatttaatcttcgtaaaatcaatatcaatcgaccatacacaaaacatgttcaaataaaatacatgggtgtacatatgtcaaccgacattgaaaaagaaaacggaataaatcagtaacaaatcaaaaaaacaaaaccataacatttctataacattccatagagtaaaaatcaacatcgaataaccttacacaaaccatacataaaccatacataaaaatacacatatgtcaaccgttaccgtaaaaagattacttaaataattttatactcacctcttattttttgtgttttttattatccagggcaacctttcccagggagaaattcttccttgcagttgcaggcaagaaccaggactttcttccagggcatactttcccagggagaaattcttccttgcagctgcaggcaagaaccaggactttcccccaggaccaggacctcaaggtctgcatccagaggaagaccttcccagggcgccaagatcccgcagattatggaacattatttcccgggaaataattcagggcttaatgcccaactgaggaatacattttaaataagcttctcttagttcatccttttctatgtggtcgtacacgtctattgcttcgcctctcgaatctccccttaattcctgaatgaatgtcctttccatacctgaacgtcgaagccatgtagtgaaccaatgccggcagcaatgcggagtaaaatgttcttcaagtctcccctcggggttatggagtccgatagaggaagcggcagatgtgacaatatcgtacactaaatttctttgtacacggtgaccgttttgtctgcctatgaataatgctttttctttatcatgcctcgtgttaaggtacgcttccaggaaggccttagtctcaaggtcaaagaatacgatacgatttgaccttttagctgtcttcttgagtctcaccgtacacttttccagatctatgtcagctcgatcaagagtaattaattcatttctcctgatgccagttttagctaaaaagattatcaatgcttgatacatggggtaacgtggtactttgacaagctttgccatgtcttcaatggtaatcaattgccttgtttccggagtatggtattttttgtagcttcttagatagcgttttctgaacgtgggaatgttgtttctctcgattatatgcttgaattcaaggaagtcaaaaaaggtactcagtgaagcaaagtaattgttacaggttgcgagtgtgtactgcctttcatctcttatgtgaacaaggaacccttcaagctccggaaaccctgcattcactccacacatttgaaggaaaaacttagcatgacttacgtatccttctattgttcttttagagtaaccccttccgatacaatcagaagtgaataaacttataagtctttcattatcaagcggttcatagataccagttagctgaatagctgccatttcacccataaatcaataaaagaagtatgtaaataaatactttgtgcatagatgtcatttaaacacaaagtataagtacatctaccttcttttagtatatgctggtaacagcaaaaaagcgccaaacaacgcatctttacccaatagaatgacgtcgattaaaagagtaagttggtggtgagccttaaaaaaggtaaaagagagcttactcttaagttaaaaacggaattggcaaacagtaaggattgttccagcagtccttactaaaaacattactttattaattctatatagtcacaacattcacaaaaataggcttttttctcgatttttccttttgaatgtttataaagaaaaatcatatagcatttattacactgtggacatattttcttttcaattttgccccttccaatttgtggaagccttttttcagtaacacgcttaggcataatgaaagatcggtgtattaagtattaacattttctaacttgtgcaataataattattcggggttgtcgaagaacttccaaacagtagaccgatccattttccacattattctaatgaagccaagactcaaggatggggggaaatatccccccacaccccccaatgagactacccaagacagcccgtatgccgacagcgacacagttaaatatcaatgtgtattaatacatatcaacaagtatcaacacacacaattaaacaataatacacatcaatggagaacaatacacatgccaacacaagaatacacacggacaaacattacattaccagaacatgttagagcaatgattaaagagtacaataaaagaagtacatggggagacctcaacgtatcagcagtatgctctaatgcaatagcagacagaattagagctacattcccaaaattagaaaaagaactatttccagaacaatttccagaacttaacccaaaaatgacaaaaaccagctcaccaggacaaccagaaataacacaaaaaggaaaatccaaacaaggagactctaaagatagaatatgcgcttattgtggccaaccttttgaaccgaaatcgcataatcaaaaattctgtaaagatgcatgtaagtcagcaaattatagaaaaaacaaaaaaacacagagttaaacccattaaccacactctttttttgaatttatctgcatttcacggtactttttatccaagaaagcccacaaatcacgctctttaaacggtttcatggaaaaattttcccggaatgcattaattaacatcttttcaattttagggttgaaataagccccataacggtaatctggcgaccaatctgaagctttaacctcttcataagtccgatacggcacattacccggaagaagcccaggaaccacaagcagataatagatatagcaccttatacacatatgtgattttttagtagtctttaaccattcaatatatgaaagatccgcacctgttcgataatcctcaaaaagaattaagttcctttgcctccgagatccacacacacagcaatacttcaattccataatacataacaccccgaaataataagacatctgaaaaaccgtatcactttaggctttaagcatcttatccggaccttcgagcccaaaagccttgttgaacgtggcatctcaatcacccccaaacatcatggagcgtgattttaagcttgtaaatagctcttcataattaatttcggaagccttttgcaagctgttagaaggcatttcaggcgattcttcaattatacagccatcatgctcccattgatgagagtcaaccaccaggaagccggcagggggatcaggaagcccttctaagaaccatacccgaagaagactcccggcaggttcaagcttcaaaaactccggtaattcatccactttgaagggagaagcccataatttacgccattttttaggaactttgtttttgccatctcgcccttttaggttatgataaaaattatttcttgcaattaattgttgcaagtaatcggcatattcaggtttaaccgctttagcttccttcaaataagcatgtagagcctgtcttccacccgaaaaatgatcccgaagctttgttaacggtatccatccagtatcatcgttttcctgtccttcccacttcaactctccattataaacgagttttacttctcgaccttcattgattaaattaaggacttcggttctggtatctatcaattgctcaaatgtcagctttccggaggaacttacaactttatcaggatgccaccggaacaaagcaccaaagcgagattctatatggatattaccgtatctgttatccgtgagctgcccaacgtgagaataaagataagtcacctgcttgtaaatatccataaggctccgcaattcccattgccttttaccattgactttatgagaaagcctctgaataaatatatcatattcatcactcttgcatctctttttactcttcggattctttccagttatacgaatgtttccagggaaaatgaaaaaatgaaaatgaggagcaaggataaggcagtcataaatagagtttatttcgatatttagcaagtccgcgatcttgtttaaattgccttcgttgtggtcgtctcttatgtaattccaaaaagcttcggagtcgtcattaccttttaaacctgttaacttacggatagcccgagttatttccggcttaatcctgtaagggtgaaagattgtagtaccagcagttacgccccagcgttccagcctgtcctttaaattcctccggaaaagtcggattgcttccacagatactccccggctaaagtccatcgaagcaaacccccatgaaggaaactgcccagaaaacagggcataagtccacaaaattacaactaatgcaaatctatgttgcaattccgataggatgccgcaaccaggacaagaaagtgtattgcagtgagaattaagatcctttcgtgcattataattttcagatacagcataagtgaaaggacgcccacagtaagcgggtctttttccctttgctggaagaacaaagtcgccaacttttagaggacgatacttagagcatttagaaaaatcgaagtcaggacaagagtagtatttacagatagaaaccccgttctttttgttgcgaaccgggcacaaaacaccatcggtacatatacacgtcgcaaaatctctacactctggagcataggaaacggcgccaggtctagaactaaaatcagaactcagagaacatttcctgtcgaaaatttaaaccgtaaaccaaagataacaattttcataatattacagaacctatattttttgtaactcgacattacaaaaatgtaccacaaaacataaaaacattttcatagaagaaatattcacaaacaatagaaaaaaattataagaataagattaacgcctaccttattaaaagaaggtgtcgagtcttcaggttccatcaaggcaggcgttgatattctatatatgttacacataacatatataacatacgtacctcagaacattcagacctgaatgctctaaaatgaaataagctttaaactttataaattcatctgctataaatagttttagagatatagaaagcccaaggaagaaaaagaatagcaaaaataaaaaagaaaaacggcccgcgacggcaaacccggagacttataatcaaagtaagacgaaaaggaagccgacaacgatttgataataacaaatcatcttttattttggcaatattcatatgatatatgaagaaatcaacatcaagcggattagtatttttgctatttgtaaatcaatatcgggtgagacagtatatcaaaaacaaacttcatttttttcgaacaaaaaaatcactatcaaatgacattgtagtaagaaactacatcaaaactagttgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacg

SEQ ID NO: 15 - DNA, 10722 bp, Artificial
  pACPmcr-IspS
gaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagcttgcatgcatcttctattttcatcttaattttatatattaaaggctaatttttcaatattttatcgcaaactattgtgttaattacatcaaatatataaacttttctatttaaagctaatgaaaaagtgaatatatacacatagagtaatgttatgatgtatatatcaaaaaaataggagtgattcctatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagttaattactaatttattattaatttattattagattgggcaaaatagtaaaagaaaactaaaggaaacctaatatggtttcctttttttatatatttttaaaaattgattatattatatgtaaacaattataattaatgttaatcgaattcaatttttatttaataaacggcgcgccacgcgtccttcaattgccgcgatcgttaattaaatgatttttaaaaaaatacatgaattcatctagcggagaacacaaaagatttaagtaccttctaaacgaatgagatttcattgggaaagtggacacttaagaacgacgcggtacttgatttattgagtgcaaaagcactcgattaggtgaccagtcccaaagtgattttaataaattaaggaggaaattaaaatggaagccagacggtctgccaattatgaaccaaatagctgggattatgattttttgctgtcttcagacactgacgaatcgattgaagtatacaaagacaaggccaaaaagctggaggctgaggtgagaagagagattaacaatgaaaaggcagagtttttgactctgcttgaactgatagataatgtccaaaggttaggattgggttaccggttcgagagtgacataaggagagccctcgacagatttgtttcttcaggaggatttgatggtgttacaaaaactagccttcatgctactgctcttagcttcaggcttctcagacagcatggctttgaggtctctcaagaagcgttcagtggattcaaggatcaaaatggcaatttcttggaaaaccttaaggaggacaccaaggcaatactaagcctatatgaagcttcatttcttgcattagaaggagaaaatatcttggatgaggccagggtgtttgcaatatcacatctaaaagagctcagcgaagaaaagattggaaaagagctggccgaacaggtgaatcatgcattggagcttccattgcatcgcaggacgcaaagactagaagctgtttggagtattgaagcataccgtaaaaaggaagatgcaaatcaagtactgctagaacttgctatattggactacaacatgattcaatcagtataccaaagagatcttcgcgagacatcaaggtggtggaggcgagtgggtcttgcaacaaagttgcattttgctaaagacaggttaattgaaagcttttactgggcagttggagttgcgttcgaacctcaatacagtgattgccgtaattcagtagcaaaaatgttttcatttgtaacaatcattgatgatatctatgatgtttatggtactctggatgagctggagctatttacagatgctgttgagagatgggatgttaacgccatcaatgatcttccggattatatgaagctctgcttcctagctctctacaacactatcaatgagatagcttatgacaatctgaaggacaagggggaaaacattcttccatacctaacaaaagcgtgggcagatttatgcaatgcattcctacaagaagcaaaatggctgtacaataagtccacaccaacatttgatgactatttcggaaatgcatggaaatcatcctcagggcctcttcaactaatttttgcctactttgccgtggttcaaaacatcaagaaagaggaaattgaaaacttacaaaagtatcatgatatcatcagtaggccttcccacatctttcgtctttgcaacgacctggcttcagcatcggctgagatagcgagaggtgaaactgcgaattccgtatcctgctacatgcgtacaaaaggcatttctgaggaacttgctactgaatccgtaatgaatttgatcgacgaaacctgtaaaaagatgaacaaagaaaagcttggtggctctttgtttgcaaaaccttttgtcgaaacagctattaaccttgcacggcaatcccattgcacttatcataacggagatgcgcatacttcaccagacgagctaactaggaaacgtgtcctgtcagtaatcacagagcctattctaccctttgagagataaactcaataaattaaacaacttaaaacagagcaggccttcgggcctgctttttctttctttcccgagtcccgcctcgggagggcggtgtcctttccgccccgaattgcgcatcgggagcacgcggtccttttcgctcgctttgctctctcaagaggactaatttacaaaattacatattgagagcgcggccgcactagtaagttatagaaatgtcatccgataccgaaaaagaaaacggaataaatcagtaacaaatcaaaaaaacaaaactataacatttaatcttcgtaaaatcaatatcaatcgaccatacacaaaacatgttcaaataaaatacatgggtgtacatatgtcaaccgacattgaaaaagaaaacggaataaatcagtaacaaatcaaaaaaacaaaaccataacatttctataacattccatagagtaaaaatcaacatcgaataaccttacacaaaccatacataaaccatacataaaaatacacatatgtcaaccgttaccgtaaaaagattacttaaataattttatactcacctcttattttttgtgttttttattatccagggcaacctttcccagggagaaattcttccttgcagttgcaggcaagaaccaggactttcttccagggcatactttcccagggagaaattcttccttgcagctgcaggcaagaaccaggactttcccccaggaccaggacctcaaggtctgcatccagaggaagaccttcccagggcgccaagatcccgcagattatggaacattatttcccgggaaataattcagggcttaatgcccaactgaggaatacattttaaataagcttctcttagttcatccttttctatgtggtcgtacacgtctattgcttcgcctctcgaatctccccttaattcctgaatgaatgtcctttccatacctgaacgtcgaagccatgtagtgaaccaatgccggcagcaatgcggagtaaaatgttcttcaagtctcccctcggggttatggagtccgatagaggaagcggcagatgtgacaatatcgtacactaaatttctttgtacacggtgaccgttttgtctgcctatgaataatgctttttctttatcatgcctcgtgttaaggtacgcttccaggaaggccttagtctcaaggtcaaagaatacgatacgatttgaccttttagctgtcttcttgagtctcaccgtacacttttccagatctatgtcagctcgatcaagagtaattaattcatttctcctgatgccagttttagctaaaaagattatcaatgcttgatacatggggtaacgtggtactttgacaagctttgccatgtcttcaatggtaatcaattgccttgtttccggagtatggtattttttgtagcttcttagatagcgttttctgaacgtgggaatgttgtttctctcgattatatgcttgaattcaaggaagtcaaaaaaggtactcagtgaagcaaagtaattgttacaggttgcgagtgtgtactgcctttcatctcttatgtgaacaaggaacccttcaagctccggaaaccctgcattcactccacacatttgaaggaaaaacttagcatgacttacgtatccttctattgttcttttagagtaaccccttccgatacaatcagaagtgaataaacttataagtctttcattatcaagcggttcatagataccagttagctgaatagctgccatttcacccataaatcaataaaagaagtatgtaaataaatactttgtgcatagatgtcatttaaacacaaagtataagtacatctaccttcttttagtatatgctggtaacagcaaaaaagcgccaaacaacgcatctttacccaatagaatgacgtcgattaaaagagtaagttggtggtgagccttaaaaaaggtaaaagagagcttactcttaagttaaaaacggaattggcaaacagtaaggattgttccagcagtccttactaaaaacattactttattaattctatatagtcacaacattcacaaaaataggcttttttctcgatttttccttttgaatgtttataaagaaaaatcatatagcatttattacactgtggacatattttcttttcaattttgccccttccaatttgtggaagccttttttcagtaacacgcttaggcataatgaaagatcggtgtattaagtattaacattttctaacttgtgcaataataattattcggggttgtcgaagaacttccaaacagtagaccgatccattttccacattattctaatgaagccaagactcaaggatggggggaaatatccccccacaccccccaatgagactacccaagacagcccgtatgccgacagcgacacagttaaatatcaatgtgtattaatacatatcaacaagtatcaacacacacaattaaacaataatacacatcaatggagaacaatacacatgccaacacaagaatacacacggacaaacattacattaccagaacatgttagagcaatgattaaagagtacaataaaagaagtacatggggagacctcaacgtatcagcagtatgctctaatgcaatagcagacagaattagagctacattcccaaaattagaaaaagaactatttccagaacaatttccagaacttaacccaaaaatgacaaaaaccagctcaccaggacaaccagaaataacacaaaaaggaaaatccaaacaaggagactctaaagatagaatatgcgcttattgtggccaaccttttgaaccgaaatcgcataatcaaaaattctgtaaagatgcatgtaagtcagcaaattatagaaaaaacaaaaaaacacagagttaaacccattaaccacactctttttttgaatttatctgcatttcacggtactttttatccaagaaagcccacaaatcacgctctttaaacggtttcatggaaaaattttcccggaatgcattaattaacatcttttcaattttagggttgaaataagccccataacggtaatctggcgaccaatctgaagctttaacctcttcataagtccgatacggcacattacccggaagaagcccaggaaccacaagcagataatagatatagcaccttatacacatatgtgattttttagtagtctttaaccattcaatatatgaaagatccgcacctgttcgataatcctcaaaaagaattaagttcctttgcctccgagatccacacacacagcaatacttcaattccataatacataacaccccgaaataataagacatctgaaaaaccgtatcactttaggctttaagcatcttatccggaccttcgagcccaaaagccttgttgaacgtggcatctcaatcacccccaaacatcatggagcgtgattttaagcttgtaaatagctcttcataattaatttcggaagccttttgcaagctgttagaaggcatttcaggcgattcttcaattatacagccatcatgctcccattgatgagagtcaaccaccaggaagccggcagggggatcaggaagcccttctaagaaccatacccgaagaagactcccggcaggttcaagcttcaaaaactccggtaattcatccactttgaagggagaagcccataatttacgccattttttaggaactttgtttttgccatctcgcccttttaggttatgataaaaattatttcttgcaattaattgttgcaagtaatcggcatattcaggtttaaccgctttagcttccttcaaataagcatgtagagcctgtcttccacccgaaaaatgatcccgaagctttgttaacggtatccatccagtatcatcgttttcctgtccttcccacttcaactctccattataaacgagttttacttctcgaccttcattgattaaattaaggacttcggttctggtatctatcaattgctcaaatgtcagctttccggaggaacttacaactttatcaggatgccaccggaacaaagcaccaaagcgagattctatatggatattaccgtatctgttatccgtgagctgcccaacgtgagaataaagataagtcacctgcttgtaaatatccataaggctccgcaattcccattgccttttaccattgactttatgagaaagcctctgaataaatatatcatattcatcactcttgcatctctttttactcttcggattctttccagttatacgaatgtttccagggaaaatgaaaaaatgaaaatgaggagcaaggataaggcagtcataaatagagtttatttcgatatttagcaagtccgcgatcttgtttaaattgccttcgttgtggtcgtctcttatgtaattccaaaaagcttcggagtcgtcattaccttttaaacctgttaacttacggatagcccgagttatttccggcttaatcctgtaagggtgaaagattgtagtaccagcagttacgccccagcgttccagcctgtcctttaaattcctccggaaaagtcggattgcttccacagatactccccggctaaagtccatcgaagcaaacccccatgaaggaaactgcccagaaaacagggcataagtccacaaaattacaactaatgcaaatctatgttgcaattccgataggatgccgcaaccaggacaagaaagtgtattgcagtgagaattaagatcctttcgtgcattataattttcagatacagcataagtgaaaggacgcccacagtaagcgggtctttttccctttgctggaagaacaaagtcgccaacttttagaggacgatacttagagcatttagaaaaatcgaagtcaggacaagagtagtatttacagatagaaaccccgttctttttgttgcgaaccgggcacaaaacaccatcggtacatatacacgtcgcaaaatctctacactctggagcataggaaacggcgccaggtctagaactaaaatcagaactcagagaacatttcctgtcgaaaatttaaaccgtaaaccaaagataacaattttcataatattacagaacctatattttttgtaactcgacattacaaaaatgtaccacaaaacataaaaacattttcatagaagaaatattcacaaacaatagaaaaaaattataagaataagattaacgcctaccttattaaaagaaggtgtcgagtcttcaggttccatcaaggcaggcgttgatattctatatatgttacacataacatatataacatacgtacctcagaacattcagacctgaatgctctaaaatgaaataagctttaaactttataaattcatctgctataaatagttttagagatatagaaagcccaaggaagaaaaagaatagcaaaaataaaaaagaaaaacggcccgcgacggcaaacccggagacttataatcaaagtaagacgaaaaggaagccgacaacgatttgataataacaaatcatcttttattttggcaatattcatatgatatatgaagaaatcaacatcaagcggattagtatttttgctatttgtaaatcaatatcgggtgagacagtatatcaaaaacaaacttcatttttttcgaacaaaaaaatcactatcaaatgacattgtagtaagaaactacatcaaaactagttgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacg

SEQ ID NO: 16 - DNA, 14004 bp, Artificial
  pACPmcr-MVA1-IDI-IspS
tttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagcttgcatgcatcttctattttcatcttaattttatatattaaaggctaatttttcaatattttatcgcaaactattgtgttaattacatcaaatatataaacttttctatttaaagctaatgaaaaagtgaatatatacacatagagtaatgttatgatgtatatatcaaaaaaataggagtgattcctatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagttaattactaatttattattaatttattattagattgggcaaaatagtaaaagaaaactaaaggaaacctaatatggtttcctttttttatatatttttaaaaattgattatattatatgtaaacaattataattaatgttaatcgaattcaatttttatttaataaacggcgcgccacgcgtccttcaattgccgcgatcgttaattaaatgatttttaaaaaaatacatgaattcatctagcggagaacacaaaagatttaagtaccttctaaacgaatgagatttcattgggaaagtggacacttaagaacgacgcggtacttgatttattgagtgcaaaagcactcgattaggtgaccagtcccaaagtgattttaataaattaaggaggaaattaaaatgaggaagagaataaaaagatcaaccggtgatttcatgtttttaaatgattatgaacttggtgaggaagaaaagcttcttttacagaaggttcttgatggagatatagcctttcgtaagatcgaagagtttgcagagccgctaacagccgtaaaaattcggagacttgctattcaggagtacgcaaaactcgaatttgaacacatccagaacttctctttagatgtagaaattgtgacaaagagaaacattgaaaacatgataggagcagttcagatccctctggggactgccggccttctgaaagtaaacggggaatatgcagacgccgaatactatatccctctggctacaacggaaggagcccttgttgccagtgtaaaccgcggatgttcggtcatcacaaagtcaggcggggcaaatgtcagagtgtttgaagatgaaatgaccagagctccggtcttcaagcttgaaagccttgacagagccaagaaattttatgaatgggtaaagcgtcccgagattttcgagcagatgaaagaggttgctgaaaagacgaccaggtttggcaagctggtttcggtaaagcctttcgttaccgggacgtatgtctatctccggttttcctatgacacaaaagatgcgatgggcatgaatatggtgaccatagccactgatgcggtgatgcacctcatagaagacgaatttggcgcgcatcccattaccctttcgagcaatatgtgcacagacaaaaaacctgcgtccataagtaccattctcgggagaggaaagaccgtggttgccgaagtaactatccctgaagagatagtcaaggagactctcaagtgtacccctgagtcaatgtttgaagtgaattacagtaaaaacctgcttggttcggcaagagccggagccatgggatttaatgcccatgctgcaaacgtcatcgctgctctttaccttgcctgcgggcaggacgctgcccacgtcgttgaaggcagcaccgcaattacgagtatggaacttacaaaatacggggagatacactgctcggtcaccctgccagctcttcctgtcggcacggtgggcggaggcaccggccttggaacccagagggattgcctcaatatccttggggtggcaggagcaggagacgagcccggcataaactcgctaaagtttgcagaaatcgtggcctctgcagtacttgcaggggagatttcccttataggggcacaggcagccggacacctggcacgcgcacatgcacaactcggacgcggaaagttctgaacaaattagttaaaaggtctaaagatgactatcggaattgtatcttacggtgcttatgtcccgagataccggataaaaattgaagaaatcgcccggctctggggtgatgacgccgaggctctcaaaaatggcctcatggtgtacgaaaagtccgtgcctgatgtcgacgaagacgcagcaacgattgcagtggaagcggcaagatatgcaatggcaagaagcggcgtagatccctccagaatcggggcggtgtacacgggttcagaaagtcatccctacgctgtaaaacctacaagcaccatcgtcgcccaggctatcggcgcaacccccgaaatgactgcagcagactttgagtttgcctgtaaagcaggaacagccgcagttcaggcctgtatgggcctcgtgggctcgggaatgatcgaccttggcctggccatcggagcagacgtttcccagggagccccaagcgatgcccttgaatacactgcagctgcaggcggagttgcctgcctgatcggaagaaaagaatccgaacttgccgcaatcattgaagatacatactcttttacaacggatacccctgacttctggagaagggaaggaatgccctatcccgaacatggaggcaggtttacgggagagcccggatacttcaagcacgtgacaaacggcgcaaaagggctgcttgaaaaactcgggacaaaacctgaagattatgattatgcagtcttccaccagccaaacgggaaattccctagcaaagctgcaaagatactcgggtttacaaaagcccagattactcccggacttgttgtcccgaaaatagggaacacctattctggttcctgcctgatgggaattgctgcaacccttgaccaggcaaagcccggagaccgtatttttgcaaccgccttcgggtcaggagcagggtccgacgcgttcagcataacagttaccgacaggattgaggaaatccgaaatagggctccgaaggtttccgaacttatcaaggatcccgtatacatcgactacgcaaggtatgccaggcataaaggcaagatccgtctggcctgaacaaattagttaaaaggtctaaagatgactgccgacaacaatagtatgccccatggtgcagtatctagttacgccaaattagtgcaaaaccaaacacctgaagacattttggaagagtttcctgaaattattccattacaacaaagacctaatacccgatctagtgagacgtcaaatgacgaaagcggagaaacatgtttttctggtcatgatgaggagcaaattaagttaatgaatgaaaattgtattgttttggattgggacgataatgctattggtgccggtaccaagaaagtttgtcatttaatggaaaatattgaaaagggtttactacatcgtgcattctccgtctttattttcaatgaacaaggtgaattacttttacaacaaagagccactgaaaaaataactttccctgatctttggactaacacatgctgctctcatccactatgtattgatgacgaattaggtttgaagggtaagctagacgataagattaagggcgctattactgcggcggtgagaaaactagatcatgaattaggtattccagaagatgaaactaagacaaggggtaagtttcactttttaaacagaatccattacatggcaccaagcaatgaaccatggggtgaacatgaaattgattacatcctattttataagatcaacgctaaagaaaacttgactgtcaacccaaacgtcaatgaagttagagacttcaaatgggtttcaccaaatgatttgaaaactatgtttgctgacccaagttacaagtttacgccttggtttaagattatttgcgagaattacttattcaactggtgggagcaattagatgacctttctgaagtggaaaatgacaggcaaattcatagaatgctataaacaaattagttaaaaggtctaaagatggaagccagacggtctgccaattatgaaccaaatagctgggattatgattttttgctgtcttcagacactgacgaatcgattgaagtatacaaagacaaggccaaaaagctggaggctgaggtgagaagagagattaacaatgaaaaggcagagtttttgactctgcttgaactgatagataatgtccaaaggttaggattgggttaccggttcgagagtgacataaggagagccctcgacagatttgtttcttcaggaggatttgatggtgttacaaaaactagccttcatgctactgctcttagcttcaggcttctcagacagcatggctttgaggtctctcaagaagcgttcagtggattcaaggatcaaaatggcaatttcttggaaaaccttaaggaggacaccaaggcaatactaagcctatatgaagcttcatttcttgcattagaaggagaaaatatcttggatgaggccagggtgtttgcaatatcacatctaaaagagctcagcgaagaaaagattggaaaagagctggccgaacaggtgaatcatgcattggagcttccattgcatcgcaggacgcaaagactagaagctgtttggagtattgaagcataccgtaaaaaggaagatgcaaatcaagtactgctagaacttgctatattggactacaacatgattcaatcagtataccaaagagatcttcgcgagacatcaaggtggtggaggcgagtgggtcttgcaacaaagttgcattttgctaaagacaggttaattgaaagcttttactgggcagttggagttgcgttcgaacctcaatacagtgattgccgtaattcagtagcaaaaatgttttcatttgtaacaatcattgatgatatctatgatgtttatggtactctggatgagctggagctatttacagatgctgttgagagatgggatgttaacgccatcaatgatcttccggattatatgaagctctgcttcctagctctctacaacactatcaatgagatagcttatgacaatctgaaggacaagggggaaaacattcttccatacctaacaaaagcgtgggcagatttatgcaatgcattcctacaagaagcaaaatggctgtacaataagtccacaccaacatttgatgactatttcggaaatgcatggaaatcatcctcagggcctcttcaactaatttttgcctactttgccgtggttcaaaacatcaagaaagaggaaattgaaaacttacaaaagtatcatgatatcatcagtaggccttcccacatctttcgtctttgcaacgacctggcttcagcatcggctgagatagcgagaggtgaaactgcgaattccgtatcctgctacatgcgtacaaaaggcatttctgaggaacttgctactgaatccgtaatgaatttgatcgacgaaacctgtaaaaagatgaacaaagaaaagcttggtggctctttgtttgcaaaaccttttgtcgaaacagctattaaccttgcacggcaatcccattgcacttatcataacggagatgcgcatacttcaccagacgagctaactaggaaacgtgtcctgtcagtaatcacagagcctattctaccctttgagagataaactcaataaattaaacaacttaaaacagagcaggccttcgggcctgctttttctttctttcccgagtcccgcctcgggagggcggtgtcctttccgccccgaattgcgcatcgggagcacgcggtccttttcgctcgctttgctctctcaagaggactaatttacaaaattacatattgagagcgcggccgcactagtaagttatagaaatgtcatccgataccgaaaaagaaaacggaataaatcagtaacaaatcaaaaaaacaaaactataacatttaatcttcgtaaaatcaatatcaatcgaccatacacaaaacatgttcaaataaaatacatgggtgtacatatgtcaaccgacattgaaaaagaaaacggaataaatcagtaacaaatcaaaaaaacaaaaccataacatttctataacattccatagagtaaaaatcaacatcgaataaccttacacaaaccatacataaaccatacataaaaatacacatatgtcaaccgttaccgtaaaaagattacttaaataattttatactcacctcttattttttgtgttttttattatccagggcaacctttcccagggagaaattcttccttgcagttgcaggcaagaaccaggactttcttccagggcatactttcccagggagaaattcttccttgcagctgcaggcaagaaccaggactttcccccaggaccaggacctcaaggtctgcatccagaggaagaccttcccagggcgccaagatcccgcagattatggaacattatttcccgggaaataattcagggcttaatgcccaactgaggaatacattttaaataagcttctcttagttcatccttttctatgtggtcgtacacgtctattgcttcgcctctcgaatctccccttaattcctgaatgaatgtcctttccatacctgaacgtcgaagccatgtagtgaaccaatgccggcagcaatgcggagtaaaatgttcttcaagtctcccctcggggttatggagtccgatagaggaagcggcagatgtgacaatatcgtacactaaatttctttgtacacggtgaccgttttgtctgcctatgaataatgctttttctttatcatgcctcgtgttaaggtacgcttccaggaaggccttagtctcaaggtcaaagaatacgatacgatttgaccttttagctgtcttcttgagtctcaccgtacacttttccagatctatgtcagctcgatcaagagtaattaattcatttctcctgatgccagttttagctaaaaagattatcaatgcttgatacatggggtaacgtggtactttgacaagctttgccatgtcttcaatggtaatcaattgccttgtttccggagtatggtattttttgtagcttcttagatagcgttttctgaacgtgggaatgttgtttctctcgattatatgcttgaattcaaggaagtcaaaaaaggtactcagtgaagcaaagtaattgttacaggttgcgagtgtgtactgcctttcatctcttatgtgaacaaggaacccttcaagctccggaaaccctgcattcactccacacatttgaaggaaaaacttagcatgacttacgtatccttctattgttcttttagagtaaccccttccgatacaatcagaagtgaataaacttataagtctttcattatcaagcggttcatagataccagttagctgaatagctgccatttcacccataaatcaataaaagaagtatgtaaataaatactttgtgcatagatgtcatttaaacacaaagtataagtacatctaccttcttttagtatatgctggtaacagcaaaaaagcgccaaacaacgcatctttacccaatagaatgacgtcgattaaaagagtaagttggtggtgagccttaaaaaaggtaaaagagagcttactcttaagttaaaaacggaattggcaaacagtaaggattgttccagcagtccttactaaaaacattactttattaattctatatagtcacaacattcacaaaaataggcttttttctcgatttttccttttgaatgtttataaagaaaaatcatatagcatttattacactgtggacatattttcttttcaattttgccccttccaatttgtggaagccttttttcagtaacacgcttaggcataatgaaagatcggtgtattaagtattaacattttctaacttgtgcaataataattattcggggttgtcgaagaacttccaaacagtagaccgatccattttccacattattctaatgaagccaagactcaaggatggggggaaatatccccccacaccccccaatgagactacccaagacagcccgtatgccgacagcgacacagttaaatatcaatgtgtattaatacatatcaacaagtatcaacacacacaattaaacaataatacacatcaatggagaacaatacacatgccaacacaagaatacacacggacaaacattacattaccagaacatgttagagcaatgattaaagagtacaataaaagaagtacatggggagacctcaacgtatcagcagtatgctctaatgcaatagcagacagaattagagctacattcccaaaattagaaaaagaactatttccagaacaatttccagaacttaacccaaaaatgacaaaaaccagctcaccaggacaaccagaaataacacaaaaaggaaaatccaaacaaggagactctaaagatagaatatgcgcttattgtggccaaccttttgaaccgaaatcgcataatcaaaaattctgtaaagatgcatgtaagtcagcaaattatagaaaaaacaaaaaaacacagagttaaacccattaaccacactctttttttgaatttatctgcatttcacggtactttttatccaagaaagcccacaaatcacgctctttaaacggtttcatggaaaaattttcccggaatgcattaattaacatcttttcaattttagggttgaaataagccccataacggtaatctggcgaccaatctgaagctttaacctcttcataagtccgatacggcacattacccggaagaagcccaggaaccacaagcagataatagatatagcaccttatacacatatgtgattttttagtagtctttaaccattcaatatatgaaagatccgcacctgttcgataatcctcaaaaagaattaagttcctttgcctccgagatccacacacacagcaatacttcaattccataatacataacaccccgaaataataagacatctgaaaaaccgtatcactttaggctttaagcatcttatccggaccttcgagcccaaaagccttgttgaacgtggcatctcaatcacccccaaacatcatggagcgtgattttaagcttgtaaatagctcttcataattaatttcggaagccttttgcaagctgttagaaggcatttcaggcgattcttcaattatacagccatcatgctcccattgatgagagtcaaccaccaggaagccggcagggggatcaggaagcccttctaagaaccatacccgaagaagactcccggcaggttcaagcttcaaaaactccggtaattcatccactttgaagggagaagcccataatttacgccattttttaggaactttgtttttgccatctcgcccttttaggttatgataaaaattatttcttgcaattaattgttgcaagtaatcggcatattcaggtttaaccgctttagcttccttcaaataagcatgtagagcctgtcttccacccgaaaaatgatcccgaagctttgttaacggtatccatccagtatcatcgttttcctgtccttcccacttcaactctccattataaacgagttttacttctcgaccttcattgattaaattaaggacttcggttctggtatctatcaattgctcaaatgtcagctttccggaggaacttacaactttatcaggatgccaccggaacaaagcaccaaagcgagattctatatggatattaccgtatctgttatccgtgagctgcccaacgtgagaataaagataagtcacctgcttgtaaatatccataaggctccgcaattcccattgccttttaccattgactttatgagaaagcctctgaataaatatatcatattcatcactcttgcatctctttttactcttcggattctttccagttatacgaatgtttccagggaaaatgaaaaaatgaaaatgaggagcaaggataaggcagtcataaatagagtttatttcgatatttagcaagtccgcgatcttgtttaaattgccttcgttgtggtcgtctcttatgtaattccaaaaagcttcggagtcgtcattaccttttaaacctgttaacttacggatagcccgagttatttccggcttaatcctgtaagggtgaaagattgtagtaccagcagttacgccccagcgttccagcctgtcctttaaattcctccggaaaagtcggattgcttccacagatactccccggctaaagtccatcgaagcaaacccccatgaaggaaactgcccagaaaacagggcataagtccacaaaattacaactaatgcaaatctatgttgcaattccgataggatgccgcaaccaggacaagaaagtgtattgcagtgagaattaagatcctttcgtgcattataattttcagatacagcataagtgaaaggacgcccacagtaagcgggtctttttccctttgctggaagaacaaagtcgccaacttttagaggacgatacttagagcatttagaaaaatcgaagtcaggacaagagtagtatttacagatagaaaccccgttctttttgttgcgaaccgggcacaaaacaccatcggtacatatacacgtcgcaaaatctctacactctggagcataggaaacggcgccaggtctagaactaaaatcagaactcagagaacatttcctgtcgaaaatttaaaccgtaaaccaaagataacaattttcataatattacagaacctatattttttgtaactcgacattacaaaaatgtaccacaaaacataaaaacattttcatagaagaaatattcacaaacaatagaaaaaaattataagaataagattaacgcctaccttattaaaagaaggtgtcgagtcttcaggttccatcaaggcaggcgttgatattctatatatgttacacataacatatataacatacgtacctcagaacattcagacctgaatgctctaaaatgaaataagctttaaactttataaattcatctgctataaatagttttagagatatagaaagcccaaggaagaaaaagaatagcaaaaataaaaaagaaaaacggcccgcgacggcaaacccggagacttataatcaaagtaagacgaaaaggaagccgacaacgatttgataataacaaatcatcttttattttggcaatattcatatgatatatgaagaaatcaacatcaagcggattagtatttttgctatttgtaaatcaatatcgggtgagacagtatatcaaaaacaaacttcatttttttcgaacaaaaaaatcactatcaaatgacattgtagtaagaaactacatcaaaactagttgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccat

(19) United States
(12) Patent Application Publication
(10) Pub. No.: US 2019/0309328 A1
(43) Pub. Date: Oct. 10, 2019

(54) RECOMBINANT CELLS AND METHOD FOR PRODUCING ISOPRENE OR TERPENE

(71) Applicant: SEKISUI CHEMICAL CO., LTD., Osaka (JP)

(72) Inventors: Masahiro FURUTANI, Arakawa-ku, Tokyo (JP); Kana MATSUSHIMA, Nagareyama-shi, Chiba (JP); Kazufumi KAWABATA, Tsukuba-shi, Ibaraki (JP)

(73) Assignee: SEKISUI CHEMICAL CO., LTD., Osaka (JP)

(21) Appl. No.: 16/334,805

(22) PCT Filed: Oct. 25, 2017
(86) PCT No.: PCT/JP2017/038551

§ 371 Date: Mar. 20, 2019

(30) Foreign Application Priority Data
Oct. 28, 2016 (JP) 2016-211836

(51) Int. Cl. C12P 5/00; C12R 1/26; C12N 1/20

(52) U.S. Cl. CPC C12P 5/007; C12R 1/26; C12N 1/20; C12Y 402/03027; C12Y 402/03102; C12Y 205/01021; C12Y 207/04026; C12Y 102/07004; C12Y 203/01009; C12Y 203/0301; C12Y 101/01088; C12Y 207/01036; C12Y 401/01033; C12Y 205/01032

(57) ABSTRACT

To provide a recombinant cell being an anaerobic archaeon, including a gene encoding isoprene synthase, a gene encoding monoterpene synthase, a gene encoding sesquiterpene synthase, a gene encoding diterpene synthase, a gene encoding squalene synthase, or a gene encoding phytoene synthase as a first foreign gene, wherein the first foreign gene is expressed, and the recombinant cell is capable of producing isoprene or terpene having 10, 15, 20, 30, or 40 carbon atoms.

### TECHNICAL FIELD

The present invention relates to a recombinant cell, and a method for producing isoprene or terpene. In particular, the present invention relates to an archael recombinant cell that produces isoprene or terpene, and a method for producing isoprene or terpene using the recombinant cell.

### BACKGROUND ART

In recent years, development of technology for manufacturing petrification alternative chemicals from syngas (mixed gas of CO, CO₂, and H₂), which is combustion gas of waste and the like using a microorganism, has been advanced. Among them, it is important to construct a petrification alternative process of isoprene as raw material of tire, and therefore, techniques for producing isoprene and terpene using a recombinant syngas assimilating bacterium belonging to the genus Clostridium have been considered (see, for example, Patent documents 1 to 3). In the conventional techniques, however, although a foreign gene for synthesizing an objective compound is expressed under a strong constitutive expression promoter, it is negatively controlled by a host cell, and therefore, only low productivity can be achieved.

Meanwhile, production of isoprene or terpene by, for example, wild-type Bacillus subtilis, recombinant E. coli, recombinant yeast, has been conventionally attempted. However, at present, the cost is still high, and practical application is difficult. Furthermore, since synthesis and metabolism of isoprene and terpene are strictly controlled in almost all organisms, in the production of isoprene and terpene using recombinant microorganisms, no examples have demonstrated production that satisfies a commercialization level. In Patent Documents 1 and 3, production of isoprene using a constitutive expression system is verified, but its productivity is very low such as about several times as that of the wild-type.

Mass-production (yield: 60 g/L) of isoprene from glucose by recombinant E. coli, into which a heterologous mevalonate pathway gene and an isoprene synthase gene have been introduced, has been demonstrated (Non-Patent Document 1). In this technique, production of isoprene from glucose is promoted by induction of IPTG. However, the process using glucose as a raw material has a problem that raw material supply is restrictive. Furthermore, since production of a substance using an inducible expression system has a low carbon converting rate (15% or less) and is carried out by not continuous expression process but batch expression process, it is not suitable for producing isoprene for commercialization.

Furthermore, it is reported that when an operon gene including a heterologous mevalonate pathway gene and an α-humulene (C15 cyclic terpene) synthase gene is constitutively expressed in Methanobacterium extroquence that is a methylotroph, a transformant is not produced due to toxicity caused by the pathway gene expression, and α-humulene is efficiently produced only by an induction expression method having strictly controlled activity (Non-Patent Document 2).

As mentioned above, production of isoprene and terpene by microorganisms has been much considered. However, there are still very few examples of productivity at practical application level. Establishment of a more efficient microorganism process is desired.

### PRIOR ART DOCUMENTS

### Patent Documents

Patent Document 1: WO2013/180584

Patent Document 2: WO2013/181647

Patent Document 3: WO2014/065271

### Non-Patent Documents

Non-Patent Document 1: Whited GM et al., Industrial biotechnology 2010, 6 (3), 152-163

Non-Patent Document 2: Sonntag F. et al., Metabolic Engineering 2015, 32, 82-94

### DISCLOSURE OF INVENTION

### Technical Problem

On the other hand, there is no specific report about techniques for producing isoprene or terpene using archaea such as natural archaea, mutant archaea, and recombinant archaea. An object of the present invention is to provide an archael recombinant cell that can efficiently produce isoprene or terpene from abundant and low-cost raw materials such as syngas, methanol, and methane, and a method for producing isoprene or terpene using the recombinant cell.

### Solution to Problem

One aspect of the present invention is a recombinant cell being an anaerobic archaeon, including a gene encoding isoprene synthase, a gene encoding monoterpene synthase, a gene encoding sesquiterpene synthase, a gene encoding diterpene synthase, a gene encoding squalene synthase, or a gene encoding phytoene synthase as a first foreign gene, wherein the first foreign gene is expressed, and the recombinant cell produces isoprene or terpene having 10, 15, 20, 30, or 40 carbon atoms.

Preferably, the recombinant cell grows using at least one selected from the group consisting of carbon monoxide, carbon dioxide, methane, methanol, and acetic acid as a sole carbon source.

Preferably, the recombinant cell includes carbon monoxide dehydrogenase.

Preferably, the recombinant cell has a function of synthesizing acetyl-CoA from methyltetrahydropterin, carbon monoxide, and CoA.

Preferably, the recombinant cell has a methane formation potential.

Preferably, the recombinant cell grows using carbon dioxide as a sole carbon source and hydrogen as an energy source.

Preferably, the recombinant cell grows using methanol as a sole carbon source.

Preferably, the recombinant cell grows using methane as a sole carbon source.

Preferably, the recombinant cell belongs to the genus Methanosarcina, the genus Methanococcus, the genus Methanothermococcus, the genus Methanothermobacter; the genus Methanothrix, the genus Thermococcus, the genus Thermofilum, or the genus Archaeoglobus.

Preferably, the recombinant cell belongs to the genus Methanosarcina, the genus Methanococcus, or the genus Methanothermococcus.

Preferably, the recombinant cell is Methanosarcina barkeri, Methanosarcina mazei, Methanosarcina acetivorans, Methanococcus voliae, Methanococcus vannielii, Methanococcus maripaludis, or Methanothermococcus (Methanococcus) thermolithotrophicus.

Preferably, the recombinant cell further includes a gene encoding carbon monoxide dehydrogenase as a second foreign gene.

Preferably, in the recombinant cell, the first foreign gene is a gene encoding isoprene synthase, the recombinant cell produces isoprene, and the isoprene synthase is derived from a plant.

Preferably, in the recombinant cell, the isoprene synthase is a protein of the following (a-1), (a-2), or (a-3):

(a-1) a protein consisting of an amino acid sequence of SEQ ID NO: 1,
(a-2) a protein consisting of an amino acid sequence in which 1 to 20 amino acids are deleted, substituted, or added in the amino acid sequence of SEQ ID NO: 1, and having isoprene synthase activity, and
(a-3) a protein consisting of an amino acid sequence having identity of 90% or more with the amino acid sequence of SEQ ID NO: 1, and having isoprene synthase activity.

Preferably, the recombinant cell further includes a gene encoding isopentenyl diphosphate isomerase as a third foreign gene.

Preferably, in the recombinant cell, the first foreign gene is a gene encoding sesquiterpene synthase, the recombinant cell produces terpene having 15 carbon atoms, and the sesquiterpene synthase is farnesene synthase.

Preferably, in the recombinant cell, the first foreign gene is a gene encoding sesquiterpene synthase, the recombinant cell produces terpene having 15 carbon atoms, and the sesquiterpene synthase is cyclic sesquiterpene synthase.

Preferably, in the recombinant cell, the farnesene synthase is a protein of the following (d-1), (d-2), or (d-3):

(d-1) a protein consisting of an amino acid sequence of SEQ ID NO: 4,
(d-2) a protein consisting of an amino acid sequence in which 1 to 20 amino acids are deleted, substituted, or added in the amino acid sequence of SEQ ID NO: 4, and having farnesene synthase activity, and
(d-3) a protein consisting of an amino acid sequence having identity of 90% or more with the amino acid sequence of SEQ ID NO: 4, and having farnesene synthase activity.

Preferably, the recombinant cell further includes a gene encoding isopentenyl diphosphate isomerase as a fourth foreign gene.

Preferably, the recombinant cell further includes at least one gene selected from the group consisting of a gene encoding geranyl diphosphate synthase and a gene encoding farnesyl diphosphate synthase, as a fifth foreign gene.

Preferably, in the recombinant cell, the first foreign gene is a gene encoding monoterpene synthase, the recombinant cell produces terpene having 10 carbon atoms, and the monoterpene synthase is cyclic monoterpene synthase.

Preferably, the recombinant cell further includes a gene encoding isopentenyl diphosphate isomerase as a sixth foreign gene.

Preferably, the recombinant cell further includes a gene encoding geranyl diphosphate synthase as a seventh foreign gene.

Preferably, the recombinant cell further includes a gene encoding neryl diphosphate synthase as an eighth foreign gene.

Preferably, in the recombinant cell, the cyclic monoterpene synthase is phellandrene synthase.

Preferably, in the recombinant cell, the phellandrene synthase is a protein of the following (b-1), (b-2), or (b-3):

(b-1) a protein consisting of an amino acid sequence of SEQ ID NO: 2;
(b-2) a protein consisting of an amino acid sequence in which 1 to 20 amino acids are deleted, substituted, or added in the amino acid sequence of SEQ ID NO: 2, and having α-phellandrene synthase; and
(b-3) a protein consisting of an amino acid sequence having identity of 90% or more with the amino acid sequence of SEQ ID NO: 2, and having α-phellandrene synthase activity.

Preferably, in the recombinant cell, the phellandrene synthase is a protein of the following (c-1), (c-2), or (c-3):

(c-1) a protein consisting of an amino acid sequence of SEQ ID NO: 3;

(c-2) a protein consisting of an amino acid sequence in which 1 to 20 amino acids are deleted, substituted, or added in the amino acid sequence of SEQ ID NO: 3, and having β-phellandrene synthase; and

(c-3) a protein consisting of an amino acid sequence having identity of 90% or more with the amino acid sequence of SEQ ID NO: 3, and having β-phellandrene synthase activity.

Preferably, in the recombinant cell, the first foreign gene is a gene encoding diterpene synthase, the recombinant cell produces terpene having 20 carbon atoms, and the diterpene synthase is cyclic diterpene synthase.

Preferably, the recombinant cell further includes a gene encoding copalyl diphosphate synthase as a ninth foreign gene.

Preferably, the recombinant cell further includes a gene encoding isopentenyl diphosphate isomerase as a tenth foreign gene.

Preferably, the recombinant cell further includes at least one gene selected from the group consisting of a gene encoding geranyl diphosphate synthase, a gene encoding farnesyl diphosphate synthase, and a gene encoding geranylgeranyl diphosphate synthase as an eleventh foreign gene.

Preferably, in the recombinant cell, the first foreign gene is a gene encoding squalene synthase, and the recombinant cell produces terpene having 30 carbon atoms. The recombinant cell further includes a gene encoding isopentenyl diphosphate isomerase as a twelfth foreign gene.

Preferably, the recombinant cell further includes at least one gene selected from the group consisting of a gene encoding geranyl diphosphate synthase and a gene encoding farnesyl diphosphate synthase as a thirteenth foreign gene.

Preferably, in the recombinant cell, the first foreign gene is a gene encoding phytoene synthase, and the recombinant cell produces terpene having 40 carbon atoms, and the recombinant cell further includes a gene encoding isopentenyl diphosphate isomerase as a fourteenth foreign gene.

Preferably, the recombinant cell further includes at least one gene selected from the group consisting of a gene encoding geranyl diphosphate synthase, a gene encoding farnesyl diphosphate synthase, and geranylgeranyl diphosphate synthase as a fifteenth foreign gene.

Preferably, the recombinant cell further includes at least one gene selected from the group consisting of acetoacetyl-CoA thiolase, HMG-CoA synthase, HMG-CoA reductase, mevalonate-5-kinase, mevalonate-5-phosphate decarboxylase, isopentenyl phosphate kinase, and isopentenyl diphosphate isomerase as a sixteenth foreign gene.

Preferably, in the recombinant cell, the first foreign gene is expressed under a constitutive expression promoter.

Another aspect of the present invention is a method for producing isoprene or terpene, the method including bringing at least one carbon source selected from the group consisting of carbon monoxide, carbon dioxide, methane, methanol, and acetic acid into contact with the above-described recombinant cell, thereby allowing the recombinant cell to produce isoprene or terpene having 10, 15, 20, 30, or 40 carbon atoms from the carbon source.

Preferably, the method includes culturing the recombinant cell using at least one carbon source selected from the group consisting of carbon monoxide, carbon dioxide, methane, methanol, and acetic acid, and obtaining isoprene or terpene having 10, 15, 20, 30, or 40 carbon atoms from the cultured product.

Preferably, in the method, a cell concentration of a culture solution of the recombinant cell in a proliferation stationary phase is 1 g or more of dry cells per liter, and the method includes continuously supplying a culture medium and discharging a culture solution.

Preferably, the recombinant cell is cultured in a culture tank, and a mixed gas consisting of carbon monoxide, carbon dioxide, and hydrogen is supplied to the culture tank.

Preferably, in the method, a carbon monoxide concentration in the mixed gas is 20% or more.

Preferably, in the method, a carbon dioxide concentration in the mixed gas is 5% or more.

Preferably, in the method, a hydrogen sulfide concentration in the mixed gas is 10 ppm or less.

Preferably, the method further include adding methanol and/or acetic acid in a culture solution.

### Effect of Invention

The present invention permits production of isoprene or terpene with high efficiency using a recombinant cell.

### BRIEF DESCRIPTION OF DRAWINGS

FIG. 1 is an explanatory diagram showing a mevalonate pathway and a membrane lipid synthesis pathway in archaea.

FIG. 2 is an explanatory diagram showing diversity of pathways in which isopentenyl diphosphate is synthesized from mevalonate.

FIG. 3 is an explanatory diagram showing a pathway of assimilation and metabolism of carbon monoxide and acetic acid by methane-generating archaea Methanosarcina acetivorans.

FIG. 4 is an explanatory diagram showing a pathway of assimilation of methanol and methane in archaea.

FIG. 5 is an explanatory diagram showing a configuration of a shuttle vector pAC:Pmcr-IDI-IspS for production of isoprene.

FIG. 6 is an explanatory diagram showing a configuration of a shuttle vector pAC:Pmcr-IDI-FnS for production of farnesene.

FIG. 7 is an explanatory diagram showing a configuration of a shuttle vector pAC:Pmcr-IDI-FnS-FPS for production of farnesene.

FIG. 8 is an explanatory diagram showing a configuration of a shuttle vector pAC:Pmcr-MVA l-IDI-IspS for production of isoprene.

### BEST MODE FOR CARRYING OUT THE INVENTION

Hereinafter, the exemplary embodiment of the present invention will be described. Note here that in the present invention, all the terms “gene” can be replaced with terms “nucleic acid” or “DNA”.

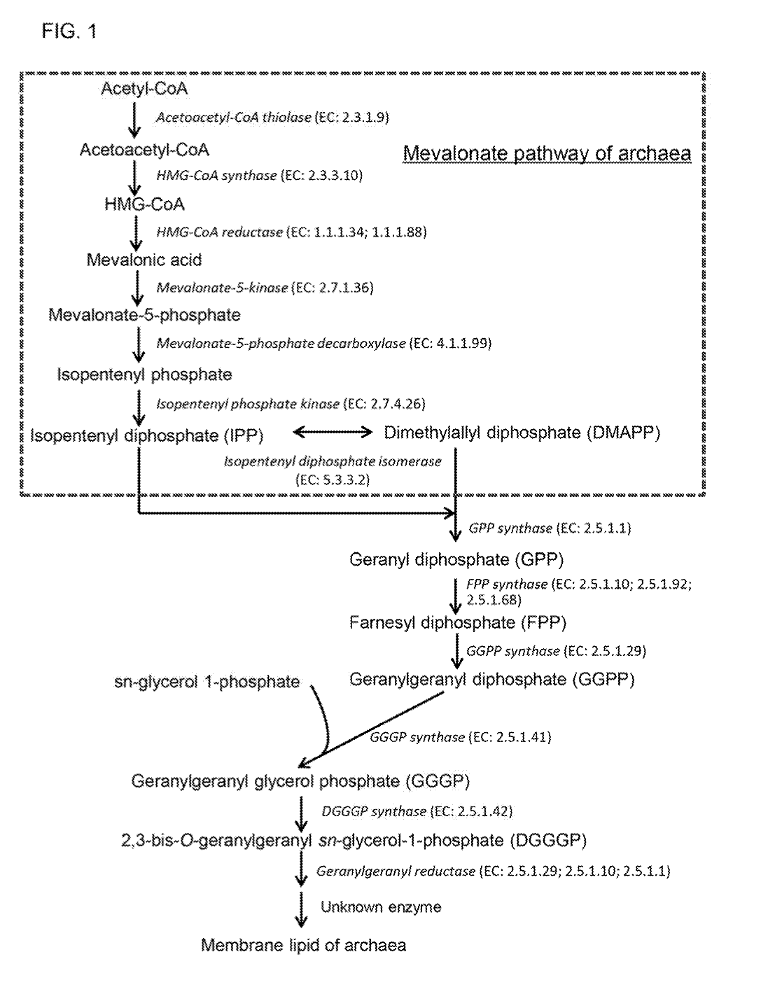

A recombinant cell of the present invention belongs to anaerobic archaea. In general, membrane lipid of archaea is ether polar lipid in which hydrocarbon (the number of carbon atoms: 20 to 40) including an isoprene skeleton is ether-linked to a glycerol skeleton. The membrane lipid peculiar to archaea is synthesized as shown in FIG. 1, in which geranylgeranyl glycerol phosphate (3-O-geranylgeranyl sn-glycerol-1-phosphate (GGGP)) is generated from geranylgeranyl diphosphate (GGPP) as a C20 compound and glycerol-1-phosphate (sn-glycerol 1-phosphate), and then two stages of reactions are carried out. The geranylgeranyl diphosphate (the number of carbon atoms: 20) is synthesized, as shown in FIG. 1, isopentenyl diphosphate (IPP) having 5 carbon atoms, geranyl diphosphate (GPP) having 10 carbon atoms, and farnesyl diphosphate (FPP) having 15 carbon atoms, which are supplied by endogenous mevalonate pathway of archaea. In this way, in archaea, unlike the other organisms, a large amount of geranylgeranyl diphosphate is always supplied through the mevalonate pathway for synthesis of membrane lipid accompanying the proliferation. The present invention uses main metabolism peculiar to archaea as described above to efficiently produce isoprene or terpene having 10 to 40 carbon atoms by a recombinant cell.

The anaerobic archaeon is referred to as an archaeon that can proliferate under the condition in which molecular oxygen is hardly present (100 ppm or less). The anaerobic archaeon in the present invention includes both strictly anaerobic one and facultative anaerobic one.

In the present invention, the recombinant cell having an isoprene synthase gene as the first foreign gene can produce isoprene. Furthermore, the recombinant cell having a monoterpene synthase gene as the first foreign gene can produce monoterpene (terpene having 10 carbon atoms). Furthermore, the recombinant cell having a sesquiterpene synthase gene as the first foreign gene can produce sesquiterpene (terpene having 15 carbon atoms). Furthermore, the recombinant cell having diterpene synthase gene as the first foreign gene can produce diterpene (terpene having 20 carbon atoms). Furthermore, the recombinant cell having a squalene synthase gene as the first foreign gene can produce triterpene (terpene having 30 carbon atoms). Furthermore, the recombinant cell having a phytoene synthase gene as the first foreign gene can produce tetraterpene (terpene having 40 carbon atoms). Hereinafter, each enzyme and each gene are described sequentially.

### Isoprene Synthase

Isoprene synthase (IspS) has action of converting dimethylallyl diphosphate (DMAPP) as an isomer of isopentenyl diphosphate (IPP) into isoprene. Note here that the structural conversion between the isopentenyl diphosphate and dimethylallyl diphosphate is catalyzed by isopentenyl diphosphate isomerase (IDI). The isopentenyl diphosphate isomerase is present in all organisms.

The isoprene synthase (IspS) used in the present invention is not particularly limited. For example, isoprene synthase derived from eukaryote such as plant can be used. General examples of the isoprene synthase derived from plants include, but not particularly limited to, isoprene synthase derived from Populus nigra, Stizolobium deeringianum, and Pueraria lobata Ohwi. Specific examples of the isoprene synthase include Q50L36, Q6EJ97, Q9AR86, Q7XAS7, A0PFK2, A0A0M4UQH9, A0A0M5MSL0 (all of the above is UniProtKB entry).

SEQ ID NO: 1 shows a nucleotide sequence of a nucleic acid (DNA) encoding the isoprene synthase derived from Populus nigra (GenBank Accession No.: AM410988.1) and a corresponding amino acid sequence. SEQ ID NO: 2 shows only the amino acid sequence.

The isoprene synthase used in the present invention may be not only a naturally occurring and isolated isoprene synthase but also a modified product thereof. For example, it may be proteins that are partial fragments of the existing isoprene synthase or may be amino acid substitution variants and have activity as isoprene synthase.

For example, the isoprene synthase used in the present invention includes at least the following protein (a-1) to (a-3):

(a-1) a protein consisting of an amino acid sequence of SEQ ID NO: 1,
(a-2) a protein consisting of an amino acid sequence in which 1 to 20 amino acids are deleted, substituted, or added in the amino acid sequence of SEQ ID NO: 1, and having isoprene synthase activity, and
(a-3) a protein consisting of an amino acid sequence having identity of 90% or more with the amino acid sequence of SEQ ID NO: 1, and having isoprene synthase activity.

Note here that the identity of an amino acid sequence in (a-3) is more preferably 92% or more, further more preferably 95% or more, and particularly preferably 98% or more.

The preferable embodiment further includes a gene encoding isopentenyl diphosphate isomerase (IDI), in addition to the isoprene synthase gene, as a foreign gene. Introduction of the IDI gene enhances the conversion from IPP to DMAPP, and can enhance the isoprene synthesis ability. The IDI used in the present invention is not particularly limited, and examples thereof include P61615, Q13907, Q46822, P50740, Q8TT35, P15496, Q10132, and Q9KWG2 (UniProtKB entry), and the like. Furthermore, in the present invention, IDI derived from archaea may be used, and, for example, it is preferable to use Q8TT35, Q8PW37, Q46CL4, A0A0E3LM81, and A0A0E3QQI3 (UniProtKB entry) derived from the genus Methanosarcina.

### Monoterpene Synthase

Monoterpene is terpene having 10 carbon atoms, consisting of two isoprene units. The monoterpene include acyclic monoterpene and cyclic monoterpene. Examples of the acyclic monoterpene include geraniol, myrcene, citral, linalool, and nerol. Examples of the cyclic monoterpene include limonene, α-phellandrene, β-phellandrene, menthol, thymol, α-pinene, β-pinene, carene, carvone, cineol, and camphor.

The monoterpene synthase is a general name of enzymes that convert geranyl diphosphate (GPP) or neryl diphosphate (NPP) into monoterpene. In a synthesis pathway of monoterpene, GPP or NPP is synthesized from isopentenyl diphosphate (IPP) by the action of the GPP synthase (GPPS) or NPP synthase (NPPS). Subsequently, monoterpene is synthesized from GPP or NPP by the action of the monoterpene synthase.

In the preferable embodiment, the monoterpene synthase is cyclic monoterpene synthase. Further preferably, the cyclic monoterpene synthase is phellandrene synthase, and, specifically, it is α-phellandrene synthase or β-phellandrene synthase.

As the a-phellandrene synthase, any enzymes can be used as long as they have activity to generate α-phellandrene from GPP or NPP as a substrate. Examples of the α-phellandrene synthase include G5CV35 and E5GAG2 (UniProtKB entry), and GN65-37361 (SolCyc GeneID), but not particularly limited thereto.

As the β-phellandrene synthase, any enzymes can be used as long they have activity to generate β-phellandrene from GPP or NPP as a substrate. Examples of the β-phellandrene synthase include Q9M7D1, C1K5M3, Q1XBU4, R9QMW3, R9QMR4, R9QMW7, E9N3U9, C0PTH8, F2XFA5, F2XFA1, F2XFA4, and A0A0B0P314 (UniProtKB entry), but not particularly limited thereto.

The monoterpene synthase used in the present invention may be not only a naturally occurring and isolated monoterpene synthase but also a modified product thereof. For example, the monoterpene synthase may be proteins that are partial fragments or amino acid substitution variants of the existing monoterpene synthase and that have monoterpene synthase activity.

For example, the phellandrene synthase (one example of the monoterpene synthase) used in the present invention includes at least the following protein (b-1) to (b-3):

(b-1) a protein consisting of an amino acid sequence of SEQ ID NO: 2,
(b-2) a protein consisting of an amino acid sequence in which 1 to 20 amino acids are deleted, substituted, or added in the amino acid sequence of SEQ ID NO: 2, and having α-phellandrene synthase activity, and
(b-3) a protein consisting of an amino acid sequence having identity of 90% or more with the amino acid sequence of SEQ ID NO: 2, and having α-phellandrene synthase activity.

Note here that the identity of the amino acid sequence in (b-3) is more preferably 92% or more, further more preferably 95% or more, and particularly preferably 98% or more.

Besides, the phellandrene synthase (one example of the monoterpene synthase) used in the present invention includes at least the following protein (c-1) to (c-3):

(c-1) a protein consisting of an amino acid sequence of SEQ ID NO: 3,
(c-2) a protein consisting of an amino acid sequence in which 1 to 20 amino acids are deleted, substituted, or added in the amino acid sequence of SEQ ID NO: 3, and having β-phellandrene synthase activity, and
(c-3) a protein consisting of an amino acid sequence having identity of 90% or more with the amino acid sequence of SEQ ID NO: 3, and having β-phellandrene synthase activity.

Note here that the identity of the amino acid sequence in (c-3) is more preferably 92% or more, further more preferably 95% or more, and particularly preferably 98% or more.

The preferable embodiment further includes a gene encoding isopentenyl diphosphate isomerase (IDI), in addition to the monoterpene synthase gene, as a foreign gene. Introduction of the IDI gene enhances the conversion from IPP to DMAPP, and can enhance the GPP synthesis ability or NPP synthesis ability. As a result, it is possible to enhance the monoterpene synthesis ability.

The preferable embodiment further includes a gene encoding GPP synthase (GPPS) or a gene encoding NPP synthase (NPPS), in addition to the monoterpene synthase gene, as a foreign gene. Introduction of these genes can enhance the synthesis ability of monoterpene from GPP or NPP. Examples of the GPPS include S4S927, S4S8D9, D8LHY4, H6VLF6, H6VLF3, D8RV97, Q6V4K1, Q8LKJ3, Q8LKJ2, Q8LKJ1, Q9FSW8, H6VLF7, V5REB1, and Q58GE8 (UniProtKB entry). Examples of the NPPS include NDPS1 derived from Solanum lycopersicum (Schilmiller AL et al., PNAS 2009, 106 (26), 10865-10870). Note here that in the case of GPPS, GPPS derived from archaea may be used, and, it is particularly preferable to use, for example, MSBRM 0487, MA_0606, MM_1767, MSBRW_3310, and Mbar_A1417 (KEGG entry) derived from the genus Methanosarcina.

### Sesquiterpene Synthase

Sesquiterpene is terpene having 15 carbon atoms, consisting of three isoprene units. The sesquiterpene includes acyclic sesquiterpene, monocyclic sesquiterpene, bicyclic sesquiterpene, and tricyclic sesquiterpene. Examples of the acyclic sesquiterpene include famesene and famesol. Examples of the monocyclic sesquiterpene include zingiberene, Humulene, and abscisic acid. Examples of the bicyclic sesquiterpene include Caryophyllene, Eudesman, Eremophilan, Valeran, Cadinan, Cadinene, Guajan, Driman, Cedrol, and Nootkatone. Examples of the tricyclic sesquiterpene include Illudan, Prezizaan, Marasman, Cedran, Thujopsan, and Hirsutan.

The sesquiterpene synthase is a general name of enzymes that convert famesyl diphosphate (FPP) into sesquiterpene. In a synthesis pathway of sesquiterpene, GPP is synthesized from IPP by the action of GPP synthase. Subsequently, FPP is synthesized from GPP by the action of FPP synthase. Subsequently, sesquiterpene is synthesized from FPP by the action of the sesquiterpene synthase.

In the preferable embodiment, the sesquiterpene synthase is cyclic sesquiterpene synthase. In another preferable embodiment, the sesquiterpene synthase is farnesene synthase.

As the famesene synthase, any enzymes can be used as long as they have activity to generate farnesene from farnesyl diphosphate (FPP) as a substrate. Examples of the famesene synthase include Q84LB2, B9RXW0, B2KSJ6, and Q84KL5 (UniProtKB entry) for synthesizing an α-form of farnesene ((3E, 6E)-alpha-farnesene), and Q9FXY7, O48935, Q2NM15, C7E5V9, C7E5V7, Q94JS8, C7E5WO, and C7E5V8 (UniProtKB entry), for synthesizing a β-form of the farnesene ((E)-beta-farnesene), but the examples are not particularly limited thereto.

The sesquiterpene synthase used in the present invention may be not only a naturally occurring and isolated sesquiterpene synthase but also a modified product thereof. For example, the sesquiterpene synthase may be proteins that are partial fragments or amino acid substitution variants of the existing sesquiterpene synthase and that have sesquiterpene synthase activity.

For example, the famesene synthase (one example of the sesquiterpene synthase) used in the present invention includes at least the following protein (d-1) to (d-3):

(d-1) a protein consisting of an amino acid sequence of SEQ ID NO: 4,
(d-2) a protein consisting of an amino acid sequence in which 1 to 20 amino acids are deleted, substituted, or added in the amino acid sequence of SEQ ID NO: 4, and having farnesene synthase activity, and
(d-3) a protein consisting of an amino acid sequence having identity of 90% or more with the amino acid sequence of SEQ ID NO: 4, and having farnesene synthase activity.

Note here that the identity of the amino acid sequence in (d-3) is more preferably 92% or more, further more preferably 95% or more, and particularly preferably 98% or more.

The preferable embodiment further includes a gene encoding IDI as a foreign gene, in addition to the sesquiterpene synthase gene. Introduction of the IDI gene can enhance the GPP synthesis ability. As a result, it is possible to enhance the FPP synthesis ability, and enhance the sesquiterpene synthesis ability.

The preferable embodiment further includes a gene encoding GPP synthase (GPPS) and/or a gene encoding FPP synthase (FPPS), in addition to the sesquiterpene synthase gene and the like, as a foreign gene. Introduction of these genes enhances synthesis ability of GPP and/or FPP, and, as a result, can enhance the synthesis ability of sesquiterpene. Examples of the GPPS include those listed above. Examples of the FPPS, include P08524, P09152, P49349, P14324, P05369, and O014230 (UniProtKB entry). Note here that FPPS derived from archaea may be used. It is particularly preferable to use, for example, MSBRM_0487, MA_0606, MM_1767, MSBRW_3310, and Mbar_A1417 (KEGG entry), derived from the genus Methanosarcina. Any one of GPPS gene and FPPS gene may be introduced, or both of them may be introduced.

### Diterpene Synthase

Diterpene is terpene having 20 carbon atoms, consisting of four isoprene units. The diterpene include acyclic diterpene, monocyclic diterpene, bicyclic diterpene, and tricyclic diterpene. Examples of the acyclic diterpene include a-tocopherol, retinol, and phytol. Examples of the cyclic diterpene include Abietane, Abietic acid, Neoabietic acid, Levomaric acid, Sapietic acid, Atisane, Beyerane, Gibbane, Gibberellic acid, Kaurane, Steviol, Labdane, Picrasane, Pimarane, Podocarpane, Rosane, Taxane, retinal, retinoic acid, and retinol.

The diterpene synthase is a general name of enzymes that convert geranylgeranyl diphosphate (GGPP) into diterpene. In a synthesis pathway of diterpene, GPP is synthesized from IPP by the action of the GPP synthase. Subsequently, FPP is synthesized from GPP by the action of the FPP synthase. Subsequently, GGPP is synthesized from FPP by the action of the GGPP synthase (GGPPS). Subsequently, diterpene is synthesized from GGPP by the action of the diterpene synthase.

As the diterpene synthase, any enzymes can be used as long as they have activity to generate diterpene from GGPP. Examples the diterpene synthase include Q38710, P9WJ61, G9MAN7, M4HY05, H8ZM70, M1VDX3, A2PZA5, Q675L5, Q0E088, P9WJ60, Q6Z5J6, and M4HYP3 (UniProtKB entry), but not particularly limited thereto.

The diterpene synthase used in the present invention may be not only a naturally occurring and isolated diterpene synthase but also a modified product thereof. For example, the diterpene synthase may be proteins that are partial fragments or amino acid substitution variants of the existing diterpene synthase and that have diterpene synthase activity.

The preferable embodiment further includes a gene encoding IDI, in addition to the diterpene synthase gene, as a foreign gene. Introduction of the IDI gene can enhance GPP synthesis ability. As a result, the FPP synthesis ability and GPP synthesis ability are strengthened, and the diterpene synthesis ability can be enhanced.

The preferable embodiment further includes at least one gene selected from the group consisting of a gene encoding GPP synthase (GPPS), a gene encoding FPP synthase (FPPS), and a gene encoding GGPP synthase (GGPPS), in addition to the diterpene synthase gene, as a foreign gene. Introduction of these genes enhances the synthesis ability of GPP, FPP, or GGPP. As a result, the diterpene.synthesis ability can be enhanced. Examples of the GPPS or the FPPS include those listed above. Examples of the GGPPS include Q12051, Q84J75, P34802, P80042, Q941137, Q9SLG2, Q9C446, Q54BK1, Q9LUE1, Q92236, Q39108, O95749, Q12051, Q9P885, and P24322 (UniProtKB entry). Note here that GGPPS derived from archaea may be used, and, for example, it is particularly preferable to use MSBRM_0487, MA_0606, MM_1767, MSBRW_3310, and Mbar_A1417, derived from the genus Methanosarcina (KEGG entry).

Any one of the GPPS gene, the FPPS gene, and the GGPPS gene may be introduced, or two or more thereof may be introduced.

The preferable embodiment further includes a gene encoding copalyl diphosphate synthase (CPPS), in addition to the diterpene synthase gene, as a foreign gene. Copalyl diphosphate (CPP) is a GGPP derivative having 20 carbon atoms. When the CPP synthase gene is introduced, a substrate of the diterpene synthase may be CPP. Examples of the CPPS include G8HZG6, O22667, A0A0N7I618, and Q0Q2G7 (UniProtKB entry).

### Squalene Synthase

Triterpene is terpene having 30 carbon atoms, consisting of six isoprene units. In general, squalene (C30) as acyclic triterpene is generated by dimerization of FPP (C15) (through catalysis of squalene synthase), 2,3-Oxidosqualene (2,3-epoxy-2,3-dihydroaqualene) is generated from squalene, and 200 types or more of triterpene skeletons can be biosynthesized through cyclization of 2,3-Oxidosqualene. However, since the generation of 2,3-Oxidosqualene from squalene has an oxygen requirement property, triterpene that can be produced by the recombinant cell that is an anaerobic archaeon of the present invention is mainly Hopene, Hopanol, and Hopanoid compounds as the derivative thereof, which are generated by the cyclization of squalene.

As described above, squalene synthase (SS) (EC 2.5.1.21) has action of dimerizing FPP. When the Hopanoid compound is synthesized, at least Squalene/Hopene cyclase (EC 5.4.99.17) gene, or Squalene/Hopanol cyclase (EC 4.2.1.129) gene, in addition to squalene synthase gene, may be introduced. In general, the Squalene/Hopene cyclase also has Squalene/Hopanol cyclization enzymatic activity. Examples of the squalene synthase (SS) include P53799, P36596, P29704, P37268, P52020, Q9HGZ6, Q9Y753, Q9SDW9, and P78589 (UniProtKB entry). Examples of the Squalene/Hopene (Squalene/Hopanol) cyclase include P33247, P33990, P54924, and P55348 (UniProtKB entry).

Further introduction of the IDI gene in addition to the SS gene can enhance the squalene synthesis ability. Furthermore, in addition, introduction of the geranyl diphosphate synthase (GPPS) gene and/or the famesyl diphosphate synthase (FPPS) gene can enhance the synthesis ability of squalene. Examples of the GPPS and FPPS are those listed above.

The squalene synthase used in the present invention may be not only a naturally occurring and isolated squalene synthase but also a modified product thereof. For example, the squalene synthase may be proteins that are partial fragments or amino acid substitution variants of the existing squalene synthase and that have squalene synthase activity.

### Phytoene Synthase

Tetraterpene is terpene having 40 carbon atoms, consisting of eight isoprene units, and mainly includes a compound group called carotenoid. Tetraterpene include many acyclic tetraterpenes or cyclic tetraterpenes. The acyclic tetraterpenes include phytoene, lycopene, and neurosporene. The monocyclic tetraterpene includes γ-carotene. The bicyclic tetraterpene includes a-carotene, β-carotene, astaxanthin, antheraxanthin, canthaxanthin, capsanthin, β-cryptoxanthin, lutein, myxoxanthophyll, zeaxanthin, fucoxanthin, rhodoxanthin, neoxanthin, and flavoxanthin.

Phytoene synthase (PYS) (EC 2.5.1.32) has action of dimerizing geranylgeranyl diphosphate (GGPP). Examples of the PYS include Q7Z859, Q9P854, P37272, Q67GH9, D5KXJ0, P21683, Q9UUQ6, P08196, B2ATBO, Q2U4X9, A2QM49, P3727I, P37273, P49085, P54975, P9WHP3, P54977, P22872, and P17056 (UniProtKB entry).

Further introduction of the IDI gene in addition to the PSY gene can enhance the phytoene synthesis ability. Furthermore, introduction of at least one gene selected from the group consisting of a GPP synthase gene, an FPP synthase gene, and a GGPP synthase gene can enhance the synthesis ability of phytoene. Examples of the GPPS, FPPS, and GGPPS are those listed above.

The phytoene synthase used in the present invention may be not only a naturally occurring and isolated phytoene synthase but also a modified product thereof. For example, the phytoene synthase may be proteins that are partial fragments or amino acid substitution variants of existing phytoene synthase and that have phytoene synthase activity.

As described above, the recombinant cell of the present invention includes an isoprene synthase gene, a monoterpene synthase gene, a sesquiterpene synthase gene, a diterpene synthase gene, a squalene synthase gene, or a phytoene synthase gene as a foreign gene, and further optionally includes an IDI gene, a GPPS gene, an NPPS gene, a GGPPS gene, a CPPS gene, an SS gene, and the like.

The above-described foreign genes employed in the present invention may be heterologous genes or homologous genes with respect to the archaeal host cell.

As already described, a membrane lipid component of archaea has an isoprene skeleton. Therefore, by an archaeal mevalonate pathway (MVA pathway) shown in FIG. 1, archaea synthesize a larger amount of IPP that is a unit structure of isoprene or an isoprenoid compound as compared with the other organisms. For producing a much larger amount of isoprene or terpene, a measures for further enhancing the IPP synthesis ability may be applied. For example, for producing a much larger amount of isoprene or terpene without affecting proliferation of the recombinant cell, an MVA pathway gene as the foreign gene may be further introduced. The MVA pathway gene to be introduced may not be particularly limited as long as it normally functions in the recombinant cell. For example, an MVA pathway gene having the same origin as the host (endogenous), an MVA pathway gene derived from the other archaea, an MVA pathway gene derived from organisms other than archaea can be used.

Furthermore, instead of introducing the MVA pathway gene from the outside, a function of an endogenous MVA pathway may be improved. For example, it is possible to increase the expression level of the endogenous MVA pathway gene by promoter modification.

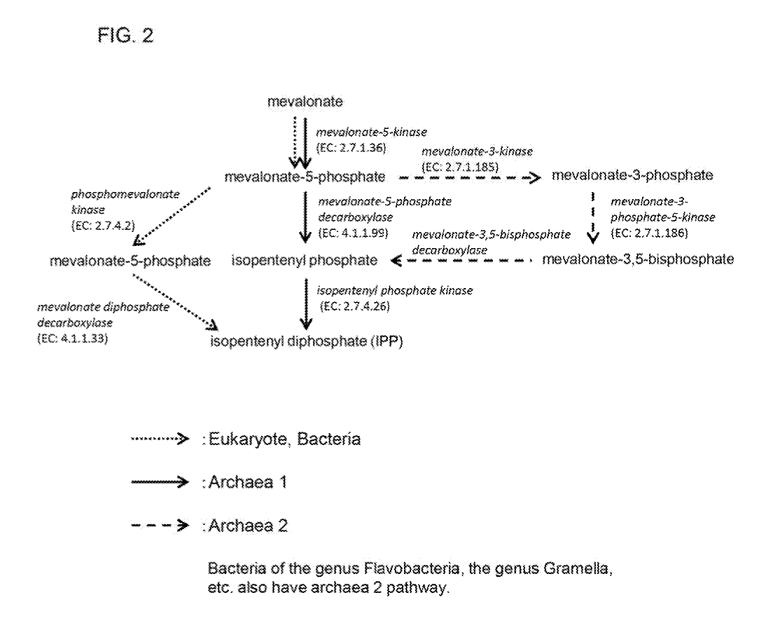

FIG. 1 shows enzymes constituting a representative MVA pathway derived from archaea, but as shown in FIG. 2, in eukaryote and prokaryote, there are various biosynthetic pathways from mevalonate to IPP. The MVA pathway genes to be introduced are genes encoding a part or whole of the enzymes shown in FIG. 1 or 2. Similarly, enzymes of the endogenous MVA pathway whose function is improved are a part or whole of these enzymes.

The enzymes shown in FIG. 1 or 2 are specifically:

acetoacetyl-CoA thiolase (EC:2.3.1.9),
HMG-CoA synthase (EC: 2.3.3.10),
HMG-CoA reductase (EC: 1.1.1.34; 1.1.1.88),
mevalonate-5-kinase (EC: 2.7.1.36),
mevalonate-5-phosphate decarboxylase (EC: 4.1.1.99),
isopentenyl phosphate kinase (EC: 2.7.4.26),
isopentenyl diphosphate isomerase (EC: 5.3.3.2),
phosphomevalonate kinase (EC: 2.7.4.2),
mevalonate diphosphate decarboxylase (EC: 4.1.1.33),
mevalonate-3-kinase (EC: 2.7.1.185),
mevalonate-3-phosphate-5-kinase (EC: 2.7.1.186), and
mevalonate-3,5-bisphosphate decarboxylase.

Among them, one or more enzymes selected from the group consisting of acetoacetyl-CoA thiolase, HMG-CoA synthase, HMG-CoA reductase, mevalonate-5-kinase, mevalonate-5-phosphate decarboxylase, and isopentenyl phosphate kinase are particularly preferable.

Furthermore, origin of the MVA pathway gene to be introduced is not particularly limited. Preferably, the MVA pathway gene is derived from archaea, and further preferably, derived from the genus Methanosarcina, the genus Methanococcus, the genus Methanothermococcus, the genus Methanothermobacter, the genus Methanothrix, the genus Thermococcus, the genus Thermofilum, the genus Thermoplasma, or the genus Archaeoglobus.

The recombinant cell of the present invention belongs to anaerobic archaea, and more specifically, is an anaerobic archaeon into which a foreign gene is introduced.

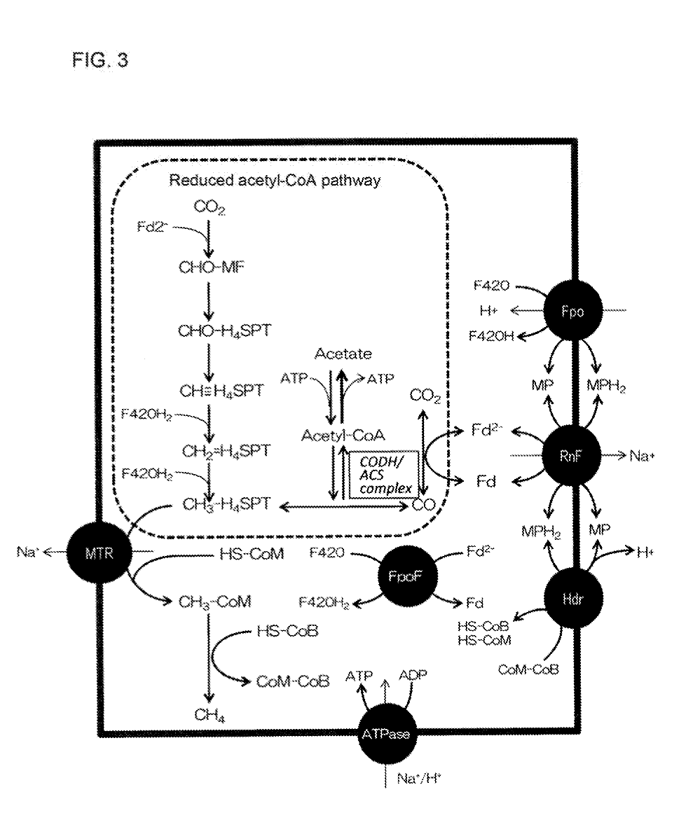

In general, the archaea can proliferate using at least one selected from carbon monoxide, carbon dioxide, methane, methanol, and acetic acid as a sole carbon source. They have a reduced acetyl-CoA pathway as shown in FIG. 3, and their basic metabolism includes synthesizing acetyl-CoA from methyltetrahydropterin, carbon monoxide, and CoA, and generating acetic acid and methane. A CODH/ACS (carbon monoxide dehydrogenase/acetyl-CoA synthase) complex has an important function in anabolism and catabolism of carbon monoxide and carbon dioxide in the reduced acetyl-CoA pathway.

Examples of archaea usable in the present invention include archaea belonging to the genus Methanobacterium, the genus Methanobrevibacter the genus Methanocalculus, the genus Methanococcus, the genus Methanosarcina, the genus Methanosphaera, the genus Methanothermobacter, the genus Methanothrix, the genus Methanoculleus, the genus Methanofollis, the genus Methanogenium, the genus Methanospirillium, the genus Methanosaeta, the genus Thermococcus, the genus Thermofilum, and the genus Archaeoglobus. Among them, the genus Methanosarcina, the genus Methanococcus, the genus Methanothermococcus, the genus Methanothermobacter, the genus Methanothrix, the genus Thermococcus, the genus Thermofilum, or the genus Archaeoglobus are preferable. Furthermore, from the viewpoint of assimilation of carbon monoxide and carbon dioxide, the genus Methanosarcina, the genus Methanococcus, or the genus Methanothermococcus is particularly preferable. Specific examples thereof include Methanosarcina barkeri, Methanosarcina mazei, Methanosarcina acetivorans, Methanococcus voltae, Methanococcus vannielii, Methanococcus maripaludis, and Methanothermococcus (Methanococcus) thermolithotrophicus.

On the other hand, carbon monoxide dehydrogenase (CO dehydrogenase, CODH) is an enzyme that is important to the carbon monoxide metabolism, and has an activity to generate carbon dioxide and proton from carbon monoxide. This activity belongs to EC 1.2 (Oxidoreductase), and specifically belongs to EC 1.2.7.4 (old EC1.2.99.2), and the like. A reaction to generate carbon dioxide and proton from carbon monoxide and water (water-gas shift reaction) is a representative example of this enzyme reaction.

In a preferable embodiment, the recombinant cell includes carbon monoxide dehydrogenase. In other words, preferably, the recombinant cell of the present invention includes the above-described enzymatic activity from the viewpoint of application to syngas fermentation. The carbon monoxide dehydrogenase may be one which a host naturally has or one expressed by a foreign gene. When a host has no or low CODH activity, it is useful to introduce a CODH gene as a foreign gene. In this case, the origin of the CODH includes, for example of bacteria, the genus Rhodopseudomonas, the genus Rhodospirillum, the genus Rubrivivax, the genus Rhodocyclus, the genus Clostridium, the genus Peptostreptococcus, the genus Acetobacterium, the genus Moorella, and the genus Carboxydothermus, which are carbon monoxide assimilation bacteria. Meanwhile, for example of archaea, the origin of the CODH includes the genus Thermococcus, the genus Thermofilum, the genus Archeoglobus, the genus Methsanosarcina, the genus Methanococcus, the genus Methanothermococcus, and the genus Methanothermobacter.

Since the recombinant cell of the present invention usually has the reduced acetyl-CoA pathway shown in FIG. 3, it can grow using carbon dioxide as a sole carbon source, and hydrogen as an energy source. Ability to proliferate by assimilating carbon dioxide and hydrogen can be applied to conversion from industrial exhaust gas, such as waste combustion gas or industrial steel and iron exhaust gas, into valuable products.

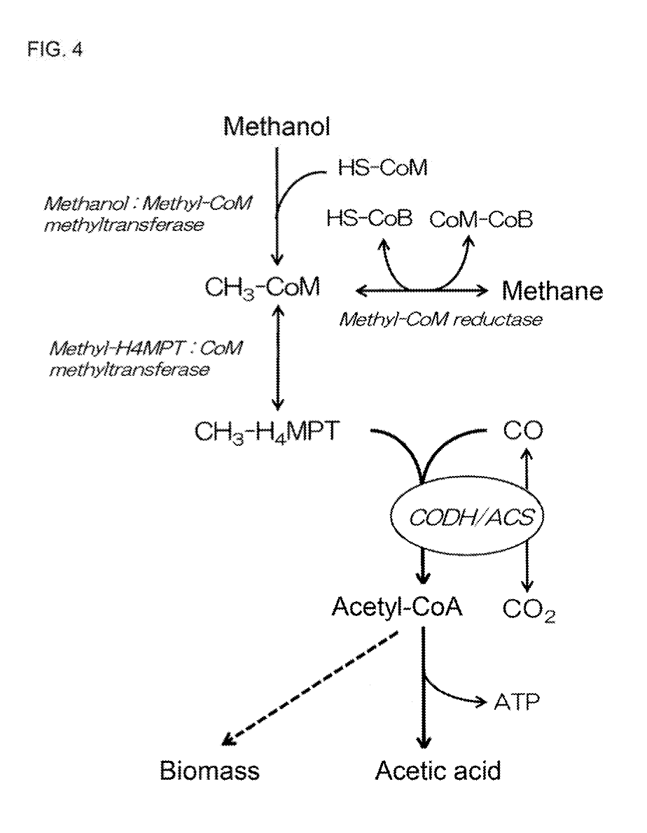

In a preferable embodiment, the recombinant cell can grow using methanol as a sole carbon source. In other words, the recombinant cells of the present invention include recombinant cells including methanol:coenzyme M methyltransferase (EC: 2.1.1.246). Since they generate methyl CoM from methanol and CoM, they can grow in a medium including methanol as a sole carbon source (FIG. 4). Methanol is produced abundantly at a low cost from natural gas, coal, waste combustion gas, and the like. Accordingly, the recombinant cell of the present invention, having methanol assimilating ability, is applicable to conversion from methanol into valuable products.

In a preferable embodiment, the recombinant cell can grow using methane as a sole carbon source. In other words, the recombinant cells of the present invention include recombinant cells including methyl coenzyme M reductase (Methyl-CoM reductase, EC2.8.4.1). Since they generate methyl CoM from methane, they can grow in a medium containing methane as a sole carbon source (FIG. 4). The recombinant cell of the present invention, having methane assimilating ability, is applicable to conversion from natural gas and biomass processed and produced methane into valuable products.

The method of introducing a gene (nucleic acid) into archaea is not particularly limited, and may be appropriately selected depending on the type of archaea as the host cell and the like. For example, it is possible to use a vector that can be introduced into the host cell and can express the introduced gene. As such a vector, it is possible to use a vector that can self-replicate in a host cell or can be introduced into chromosome and that contains a promoter at the position allowing transcription of the incorporated gene. For example, it is preferable to construct a series of constitutions including a promoter, a ribosome binding sequence, the above-described foreign gene(s) and a transcription termination sequence, in the host cell, by using the vector.

A case where the host cell is the genus Methanosarcina is described. For example, a shuttle vector with E. coli based on a plasmid pC2A that is included in the Methanosarcina acetivorans (DSM2834) can be used (Sowers K. R. et al., J. Bacteriol. 1988, 170, 4979-4982; Metcalf W. W. et al., PNAS 1997, 94, 2626-2631). Examples of introduction and deletion of gene by homologous recombination are disclosed (Rother M., et al., J. Bacteriol 2005, 187, 5552-5559; Conway D. M., J. Mol. Biol. 1996, 262, 12-20). These techniques can be used also in the present invention.

When the host cell is the genus Methanococcus or the genus Methanothermococcus, a shuttle vector can be constructed from a plasmid pURB500, pURB900, and the like, included in Methanococcus maripaludis, Methanococcus st. AG83, and the like (Lange M. et al., FEMS Microbiol. 2001, 25, 553-571).

As a promoter for expressing a foreign gene, a constitutive expression promoter can be used. The constitutive expression promoter is not particularly limited. However, since most of archaea have methane formation potential, for example, a Methyl Co-Enzyme M reductase promoter (Pmcr) can be used (Buan N. et al., Methods in Enzymology 2011, 494, 23-42). On the other hand, as the inducible expression system, a technique using a control system of expression of tetracycline-resistant gene derived from Tn10 transposon (Guess A. M. et al., Archaea 2008, 2, 193-203), and the like can be used.

For introducing a plurality of foreign genes into the host by using a vector, the genes may be incorporated into one vector, or incorporated in different vectors. When a plurality of genes is incorporated into one vector, these genes may be expressed under a common promoter, or expressed under different promoters. To express a plurality of enzymes in a form of a fusion protein, a fused gene in which a plurality of genes is linked may be introduced.

The transformation method is not particularly limited, and, for the genus Methanosarcina, a liposome method (Metcalf W W. et al., PNAS 1997, 94, 2626-2631) is useful. Furthermore, for the genus Methanococcus or the genus Methanothermococcus, a PEG method (Gernhardt P. et al., Mol. Gen. Genet. 1990, 221, 273-279) is effective.

In addition to the above-described foreign gene introduction, when mutation or cell fusion is further carried out, a strain in which productivity of isoprene and terpene is especially improved can be bred.

The method for producing isoprene or terpene of the present invention includes: bringing at least one carbon source selected from the group consisting of carbon monoxide, carbon dioxide, methane, methanol, and acetic acid into contact with the above-described recombinant cell; and allowing the recombinant cell to produce isoprene or terpene having 10, 15, 20, 30, or 40 carbon atoms from the carbon source. Typically, the recombinant cell is cultured using at least one carbon source selected from the group consisting of carbon monoxide, carbon dioxide, methane, methanol, and acetic acid, and isoprene or terpene having 10, 15, 20, 30, or 40 carbon atoms is obtained from the cultured product.

Since the recombinant cell of the present invention usually includes a reduced acetyl-CoA pathway, further, a methanol assimilation pathway, a reversible acetic acid synthesis pathway, and reversible methane production pathway, isoprene or terpene can be produced using at least one carbon source selected from the group consisting of carbon monoxide, carbon dioxide, methane, methanol, and acetic acid. The C1 compound or acetic acid may be used singly or in combination of two or more thereof.

When a main carbon source and an energy source are a gas component, culturing under a pressurized condition at about 0.2 to 0.3 MPa (absolute pressure), preferably, enhances the assimilation property of the gas component. Furthermore, for improving initial proliferation and attained cell density, small amounts of organic substances such as vitamins, yeast extract, corn steep liquor, and Bacto Tryptone, may be added.

Isoprene or terpene may be produced without culturing. In other words, isoprene or terpene can be produced by bringing the above-mentioned C1 compound or acetic acid into contact with the recombinant cell regardless of whether or not cell division (cell proliferation) occurs. For example, the above-mentioned C1 compound or acetic acid is continuously supplied to the immobilized recombinant cell, so that the isoprene or terpene can be continuously produced. Also in this aspect, the C1 compound or acetic acid as a carbon source thereof may be used singly or in combination of two or more thereof. Furthermore, it is preferable that hydrogen (H2) as an energy source is brought into contact concurrently.

In a preferable embodiment, the recombinant cell is cultured in a culture tank, a mixed gas including carbon monoxide, carbon dioxide, and hydrogen is supplied to the culture tank. Preferably, a carbon monoxide concentration in the mixed gas is 20% or more. Preferably, a carbon dioxide concentration in the mixed gas is 5% or more. Preferably, a hydrogen sulfide concentration in the mixed gas is 10 ppm or less.

In a case where the recombinant cell of the present invention is cultured using the mixed gas, addition of methanol and/or acetic acid in a culture medium may stabilize culturing. For example, the recombinant cell of the present invention includes cells allowing both chemoautotrophic growth (CO/CO₂/H₂ assimilation) and heterotrophic growth (methanol assimilation). In this case, efficient and stable culture using the following combinations of a carbon source and an energy source can be carried out.

CO/CO₂/H₂/methanol

### CO/H₂/methanol

### CO/methanol

CO₂/H₂/methanol

### CO₂/methanol

CO/CO₂/H₂/acetic acid
CO/H₂/acetic acid
CO/acetic acid
CO₂/H₂/acetic acid
CO₂/acetic acid

The gas components (CO, CO₂, and H₂) and the fluid components (methanol and acetic acid) mentioned above may be always allowed to coexist, or used separately in each stage of culture. For example, cells may be allowed to proliferate with only methanol until a stationary state, and thereafter, may be changed to proliferation with only the gas component, or a combination of the gas components and methanol. When methanol is allowed to coexist in fermentation of the syngas (mixed gas of CO, CO₂, and H₂), syngas assimilation in long-term continuous culture can be stabilized. Reduction of contamination by methanol can be expected.

The present invention is applicable to recycling of waste combustion exhaust gas, steel industry exhaust gas, and the like. In other words, a mixed gas mainly including carbon monoxide (CO), carbon dioxide (CO₂), and hydrogen (H₂) as main carbon and energy component in the above-described exhaust gas is supplied to the recombinant cell. In other words, a recombinant cell is cultured using the mixed gas as a carbon source, or a mixed gas is brought into contact with the recombinant cell to produce isoprene or terpene from carbon monoxide or carbon dioxide in the mixed gas. Also in this case, hydrogen is used as an energy source. The composition of the main component in the above-mentioned industrial exhaust gas is, for example, CO/CO₂/H₂=30-50/10-40/25-45% for waste combustion waste gas, and for example, CO/CO₂/H₂=50-70/10-40/2-10% for steel industry waste gas.

The above-mentioned composition of gas may be changed corresponding to the gas assimilating property of archaea to be used. For example, carbon monoxide in the above-mentioned mixed gas can be easily converted into carbon dioxide and hydrogen in the presence of water vapor by, for example, a water gas shift reaction so that the concentration of carbon dioxide and hydrogen can be increased.

Furthermore, hydrogen sulfide is present in the industrial waste gas, and, usually, exposure to 1000 ppm or more may dramatically deteriorate proliferation of archaea. Therefore, before the waste gas is supplied to the culture tank, the hydrogen sulfide concentration is reduced preferably to 100 ppm or less, and further preferably 10 ppm or less, by a wet limestone-gypsum process, a hydrodesulfurization process, and the like.

In a preferable embodiment, a cell concentration in the culture solution in the proliferation stationary phase is 1 g dry cells/liter or more, and supplying of a culture medium and discharging of a culture solution are continuously carried out. In other words, the culture method is continuous culture method and cell density is kept high.

The continuous culture means that after the cell density reaches a certain level, supply of a fresh culture medium and gas, and discharge of a culture solution (including a cell) are carried out continuously so as to keep the cell density and the production rate of a target constant, and to carry out culture over a long period of time (for example, one month or more). Thus, as compared with batch production, productivity of isoprene or terpene can be increased especially, and furthermore, isoprene or terpene can be produced at a lower cost. The cell density in the above-mentioned continuous culture is usually 1 g dry cells/liter or more, preferably, 5 g dry cells/liter, and further preferably, 10 g dry cells/liter.

The produced isoprene or terpene can be collected from the outside of the cells, that is, from a culture solution or a vapor phase fraction.

In the following, the present invention will be described more specifically by way of Examples. However, the present invention is not limited to these Examples.

### EXAMPLES

### Example 1

### Preparation of Isoprene Synthesis Gene Introduction Vector for Producing Isoprene in Archaea

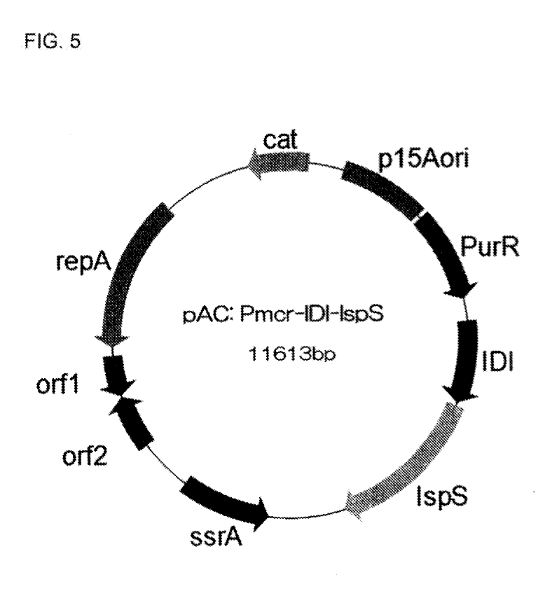

In this Example, a vector pAC:Pmcr-IDI-IspS for synthesizing isoprene in archaea shown in FIG. 5 was prepared. This vector is a shuttle vector having a replication region of an endogenous plasmid in Methanosarcina acetivorans, and a replication region p15A in E. coli, and can constitutively express an IDI (isopentenyl diphosphate isomerase) gene derived from Saccharomyces cerevisiae, and an IspS (isoprene synthase) gene derived from Poplus canescens by a promoter PmcrB (methyl coenzyme M reductase) derived from Methanococcus voltae.

A plasmid pACYC184 (manufactured by NIPPON GENE CO., LTD.) was cleaved by HindIII and BcII to obtain fragments including a chloramphenicol-resistant gene (Cat) and replication region p15A, and a linker sequences consisting of SEQ ID NO: 5 and SEQ ID NO: 6 are introduced so as to obtain plasmid pAMCS. A puromycin-resistant gene (PuR, manufactured by Genewiz) of SEQ ID NO: 7 was introduced into a SphI-AscI cleavage site of pAMCS to construct pAY:PuR. A gene fragment including an IDI-IspS gene of SEQ ID NO: 8 (manufactured by Genewiz) was introduced into a PacI-NotI cleavage site of this plasmid vector to construct a plasmid pAY:Pmcr-IDI-IspS.

On the other hand, an endogenous plasmid pC2A (GenBank No. U78295) was prepared from cultured cells of M. acetivorans (DSM2834) according to the method by Sowers KR et al. (1988 J. Bacterial., 170 (10), 4979-4982). pC2A linearized by SpeI and pAY:Pmcr-IDI-IspS linearized by SpeI were fused to construct a shuttle vector pAC:Pmcr-IDI-IspS (SEQ ID NO: 9, FIG. 5) for constitutively expressing the genes for isoprene synthesis in archaea.

pAY:PuR and pC2A were fused by the similar procedure so as to prepare a shuttle vector pAC:PuR (SEQ ID NO: 10) for control.

### Example 2

### Transformation of Methanosarcina barker, and Production of Isoprene from VARIOUS CARBON SOURCES

Single cell-forming culture of Methanosarcina barker (DSM800) and preparation of a competent cell were carried out according to the method of Metcalf WW et al. (PNAS 1997, 94, 1997). In other words, an ATCC2825 modified culture medium (10 g of magnesium chloride hexahydrate, 0.76 g of potassium chloride, and 24 g of sodium chloride per 1 L) (pH6.8) was cultured under strict anaerobic conditions until the logarithmic growth phase (OD600=0.3−0.6). Cells were collected in an anaerobic chamber (manufactured by Coy) by centrifugation. The collected cells were suspended in 0.85 M saccharose aqueous solution to obtain a competent cell suspension (about 10⁹ cells/mL).

Fifteen microliters (μL) of DOTAP liposome (manufactured by Boehringer Mannheim), 100 μL of 20 mM HEPES-KOH (pH7.4), and 2 μg of pAC:Pmcr-IDI-IspS (in 50 μL of HEPES-KOH (p117.4)) were mixed, and the mixture was incubated for 15 minutes. One microliter (mL) of the competent cell suspension was added thereto, and the mixture was further incubated for four hours. This mixture solution (1.165 mL) was recovery cultured in 10 mL of the ATCC2825 modified culture medium at 35° C. for 12 to 16 hours, and then the cells were collected. The collected cells were suspended in 2 mL of ATCC2825 modified culture medium (45° C.) including 0.5% agarose, and the suspension was seeded in ATCC2825 modification agar culture medium including 1.4% agarose and puromycin (2 μg/mL). Culture in the agar culture medium was carried out in an anaerobic jar, in which a vapor phase had been substituted with a mixed gas having N₂/CO₂/H₂S=80/20/0.1% at 35° C. for 10 to 14 days.

Growing colony was cultured at 35° C. for three days using 200 μL of ATCC2825 modified culture medium (wherein 500 mg of yeast extract, 500 mg of Casitone, and 5 g of sodium chloride per liter were included, and methanol was not included) and a predetermined carbon source. Cells in a whole culture solution were subjected to shaking culture at 35° C. for 7 to 10 days under five types of conditions shown in Table 1. In experiments 1 to 4, various gases were filled at a pressure of 2.5 atmospheric pressure (absolute pressure). In experiments 5 and 6, N₂ was used as the vapor phase, and gases were filled at a pressure of 1.5 atmospheric pressure (absolute pressure). Culture was carried out in a 20 mL-vial for HS-20 sealed with a butyl rubber plug (vapor phase: 15 mL, and culture solution: 5 mL). In each experiment, three clones each was cultured (N=3). After the culture was completed, a vapor phase was subjected to a head-space GCMS analysis (GCMS-QP2010 Ultra/HS-20, manufactured by SHIMADZU CORPORATION), and the isoprene concentration was measured.

The similar operation was carried out using pAC:PuR instead of pAC:Pmcr-IDI-IspS as a control.

The results are shown in Table 1. When pAC:Pmcr-IDI-IspS (FIG. 5) was introduced, in any case using any carbon sources of CO₂/H₂ (Experiment 1), CO/CO₂/H₂ (Experiment 2), and CO/H₂/N₂ (Experiment 3), significant isoprene production was observed. Furthermore, also when methanol (Experiment 4) and acetic acid (Experiment 5) were used as a carbon source, significant isoprene production was observed. A production amount of isoprene exceeded 1 mg/g dry cell in all of these cases. On the other hand, when a main carbon source is not included (Experiment 6), only a small amount of isoprene was produced. Note here that when pAC:PuR was introduced, isoprene was hardly detected.

From the above description, production of isoprene derived from all of carbons, i.e., CO, CO₂, methanol, and acetic acid by recombinant archaea was observed.

It was shown that the production amount of isoprene (more than 1 mg per 1 g dry cell) was clearly higher than a previously reported production amount of isoprene by a syngas assimilating Clostridium bacterium (see, for example, Patent Documents 1 and 3), and that isoprene production in archaea was efficient. Furthermore, it was shown that since isoprene was produced using CO/CO₂/H₂, and CO₂/H₂ as a carbon source, syngas such as waste combustion gas was able to be industrially used as a carbon source.

Furthermore, in conventional techniques using bacteria as a host, including the investigation example in the genus Clostridium descried above, it had been necessary to introduce an MVA pathway gene constructed by six or more types of foreign enzyme genes. That is, also from the viewpoint of easiness in gene manipulation, an endogenous MVA pathway gene of archaea can be used. In other words, it was shown that the present invention, which does not require a foreign MVA pathway gene, was effective.

As described above, it was also shown that the present invention was applicable to production of isoprene from waste combustion gas, methanol, acetic acid, and the like, being manufactured at a low cost and abundantly.

TABLE 1

| | | Isoprene production | |
|---|---|---|---|
| | | (mg/g dry cell) | |
| Experiment | Carbon source | pAC:Pmcr-IDI-IspS | pAC:PuR |
| 1 | CO₂/H₂ = 20/80 | 9.8 | <0.01 |
| 2 | CO/CO₂/H₂ = 15/30/55 | 6.8 | <0.01 |
| 3 | CO/H₂/N₂ = 15/50/35 | 4.3 | <0.01 |
| 4 | 250 mM CH₃OH | 10.8 | <0.01 |
| 5 | 15 mM CH₃COONa | 8.6 | <0.01 |
| 6 | None | <0.1 | <0.01 |

### Example 3

### Preparation of Vector for Production of Farnesene in Archaea

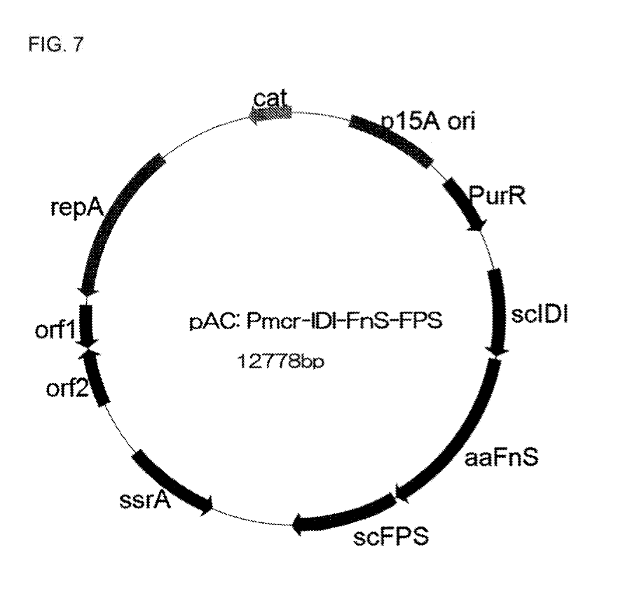

In this Example, vectors pAC:Pmcr-IDI-FnS (Fig_{—} 6) and pAC:Pmcr-IDI-FnS-FPS (FIG. 7) for synthesizing farnesene in archaea were prepared. The pAC:Pmcr-IDI-FnS contains IDI derived from Saccharomyces cerevisiae and Farnesene synthase (FnS) gene derived from Artemisia annua. The pAC:Pmcr-IDI-FnS-FPS contains the above-mentioned IDI gene, farnesyl diphosphate synthase (FPS) gene derived from Saccharomycess cerevisiae, and the above-mentioned FnS gene.

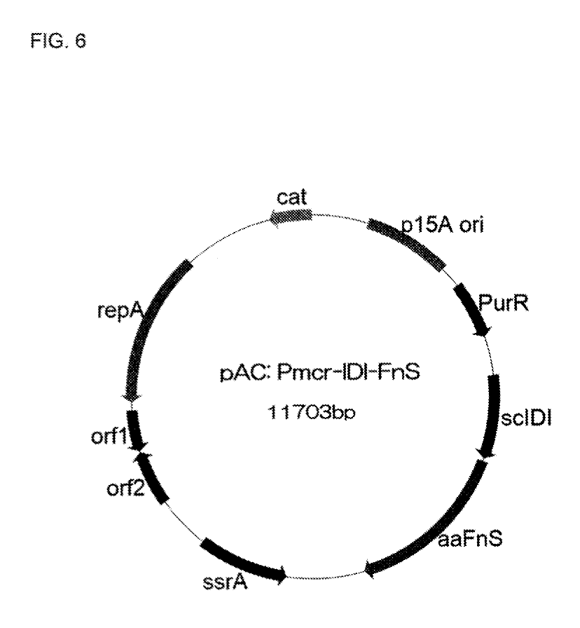

The pAC:Pmcr-IDI-IspS (FIG. 5) prepared in Example 1 was cleaved by MluI-NotI to delete an IDI-IspS region, and a linear fragment was obtained. This linear fragment and a gene fragment containing IDI-FnS (SEQ ID NO: 11) were fused to construct pAC:Pmcr-IDI-FnS (SEQ ID NO: 13, FIG. 6). Furthermore, this linear fragment and a gene fragment containing IDI-FnS-FPS gene (SEQ ID NO: 12) (manufactured by Genewiz) were fused to construct pAC:Pmcr-IDI-FnS-FPS (SEQ ID NO: 14, FIG. 7). In FIGS. 6 and 7, scIDI represents IDI derived from Saccharomyces cerevisiae, aaFnS represents FnS derived from Artemisia annua, and scFPS represents farnesyl diphosphate synthase derived from Saccharomycess cerevisiae.

### Example 4

### Production of Farnesene from Various Carbon Sources by Recombinant Methanosarcina barkeri

By transformation method by liposome method similar to Example 2, pAC:Pmcr-IDI-FnS or pAC:Pmcr-IDI-FnS-FPS were introduced into Methanosarcina barkeri (DSM800) to obtain transformants for each. Subsequently, these transformants were cultured in the same conditions as in Example 2. However, for extracting and absorbing farnesene to be produced, 10% (v/v) dodecane was overlaid on a culture solution. After culture was completed, a dodecane layer was collected from each culture solution by centrifugation. The collected dodecane layer was 10-folded diluted with n-hexane, farnesene concentration was measured by GCMS (GCMS-QP2010 Ultra, manufactured by SHIMADZU CORPORATION) analysis.

The similar operation was carried out as a control using pAC:PuR (Example 1) instead of pAC:Pmcr-IDI-FnS or pAC:Pmcr-IDI-FnS-FPS.

The results are shown in Table 2. When pAC:Pmcr-IDI-FnS (FIG. 6) or pAC:Pmcr-IDI-FnS-FPS (FIG. 7) was introduced, in any case using any carbon/energy source of CO₂/H₂ (Experiment 7), CO/CO₂/H₂ (Experiment 8), and CO/H₂/N₂ (Experiment 9), significant farnesene production was observed. Furthermore, also when methanol (Experiment 10) or acetic acid (Experiment 11) was used as a carbon source, significant farnesene production was observed. These production amounts of the farnesene exceeded 1 mg/g dry cell.

On the other hand, even when pAC:Pmcr-IDI-FnS (FIG. 6) or pAC:Pmcr-IDI-FnS-FPS (FIG. 7) was introduced, if a main carbon source was not present (Experiment 12), only a slight amount of farnesene was produced. It was shown that since farnesene was able to be produced using CO/CO₂/H₂, CO₂/H₂, and CO/H₂ as a carbon source, the present invention was applicable to production of farnesene from syngas such as waste combustion gas.

Note here that when pAC:PuR was introduced, in all cases, farnesene was hardly detected.

As described above, it was also shown that the present invention was applicable to production of farnesene from waste combustion gas, methanol, acetic acid, and the like, being manufactured at a low cost and abundantly.

TABLE 2

| | | Famesene production (mg/g dry cell) | | |
|---|---|---|---|---|
| Experiment | Carbon source | pAC:Pmcr-IDI-FnS | pAC:Pmcr-IDI-FnS-FPS | pAC:PuR |
| 7 | CO₂/H₂ = 20/80 | 7.8 | 9.9 | <0.01 |
| 8 | CO/CO₂/H₂ = 15/30/55 | 5.8 | 6.6 | <0.01 |
| 9 | CO/H₂/N₂ = 15/50/35 | 3.3 | 5.1 | <0.01 |
| 10 | 250 mM CH₃OH | 7.7 | 10.4 | <0.01 |
| 11 | 15 mM CH₃COONa | 5.6 | 6.7 | <0.01 |
| 12 | None | <0.1 | <0.1 | <0.01 |

### Example 5

### Influence on Foreign Isopentenyl Diphosphate Isomerase (IDI) Gene in Production of Isoprene by Recombinant Methanosarcina barker

A vector pAC:Pmcr-IspS (SEQ ID NO: 15) was prepared. This vector corresponds to one in which an IDI gene is deleted from pAC:Pmcr-IDI-IspS (FIG. 5).

pAC:Pmcr-IspS or pAC:Pmcr-IDI-IspS was introduced into Methanosarcina barkeri (DSM800) by the same method as in Example 2 to obtain each transformant. Each transformant was cultured in the same conditions as in Example 2, and the concentration of isoprene in the vapor phase was measured. CO₂/H₂ (Experiment 13) or methanol (Experiment 14) was used as a carbon source for culture.

The results are shown in Table 3. With any carbon sources, the production amount of isoprene was higher when the IDI gene was introduced. This showed that when the IDI was allowed to act concurrently, the production amount of isoprene was increased.

TABLE 3

| | | Isoprene production (mg/g dry cell) | |
|---|---|---|---|
| Experiment | Carbon source | pAC:Pmcr-IDI-IspS | pAC:Pmcr-IspS |
| 13 | CO₂/H₂ = 20/80 | 10.2 | 6.4 |
| 14 | 250 mM CH₃OH | 13.4 | 8.3 |
| 15 | None | <0.1 | <0.1 |

### Example 6

### Enhancement of Isoprene Production by Introduction of MVA Pathway Enzyme Gene

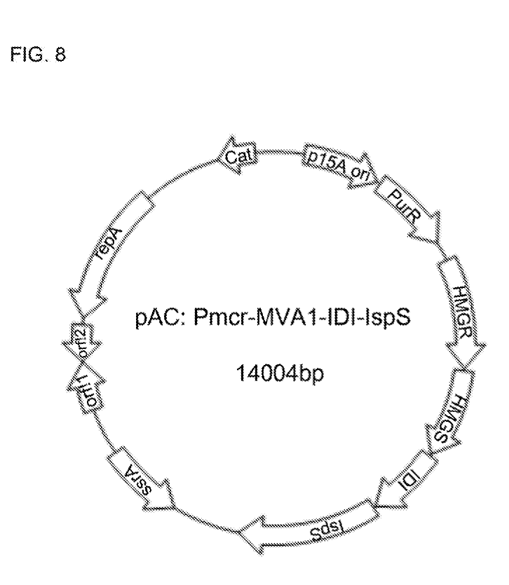

A vector pAC:Pmcr-MVA1-IDI-IspS (SEQ ID NO: 16, FIG. 8) was prepared. This vector further includes HMG-CoA synthase (KEGG entry: MA_4041) gene derived from Methanosarcina acetivotans and an HMG-CoA reductase (MA_3073) gene in addition to an IDI gene derived from Saccharomyces cerevisiae (Example 1) and an IspS gene derived from Poplus canescens (Example 1).

pAC:Pmcr-MVA1-IDI-IspS or pAC:Pmcr-IDI-IspS (Example 1) was introduced into Methanosarcina barkeri (DSM800) by the same method as in Example 2 to obtain each transformant. Each transformant was cultured in the same conditions as in Example 2, and the concentration of isoprene in the vapor phase was measured. CO₂/H₂ (Experiment 16) or methanol (Experiment 17) was used as a carbon source for culturing.

The results are shown in Table 4. With any carbon sources, the production amount of isoprene was higher in the transformant in which pAC:Pmcr-MVA1-IDI-IspS had been introduced. This showed that only reinforcing a part of enzymes constructing an MVA pathway increased the production amount of isoprene.

TABLE 4

| | | Isoprene production (mg/g dry cell) | |
|---|---|---|---|
| | | pAC:Pmcr- | |
| Experiment | Carbon source | MVA1-IDI-IspS | pAC:Pmcr-IDI-IspS |
| 16 | CO₂/H₂ = 20/80 | 13.2 | 9.9 |
| 17 | 250 mM CH₃OH | 17.3 | 12.8 |
| 18 | None | <0.1 | <0.1 |

1. A recombinant cell being an anaerobic archaeon, comprising: a gene encoding isoprene synthase, a gene encoding monoterpene synthase, a gene encoding sesquiterpene synthase, a gene encoding diterpene synthase, a gene encoding squalene synthase, or a gene encoding phytoene synthase as a first foreign gene,
wherein the first foreign gene is expressed, and the recombinant cell produces isoprene or terpene having 10, 15, 20, 30, or 40 carbon atoms.

2. The recombinant cell according to claim 1, growing using at least one selected from the group consisting of carbon monoxide, carbon dioxide, methane, methanol, and acetic acid as a sole carbon source.

3. The recombinant cell according to claim 1, comprising carbon monoxide dehydrogenase.

4. The recombinant cell according to claim 1, having a function of synthesizing acetyl-CoA from methyltetrahydropterin, carbon monoxide, and CoA.

5. The recombinant cell according to claim 1, having a methane formation potential.

6. The recombinant cell according to claim 1, growing using carbon dioxide as a sole carbon source and hydrogen as an energy source.

7. The recombinant cell according to claim 1, growing using methanol as a sole carbon source.

8. (canceled)

9. The recombinant cell according to claim 1, belonging to the genus Methanosarcina, the genus Methanococcus, the genus Methanothermococcus, the genus Methanothermobacter, the genus Methanothrix, the genus Thermococcus, the genus Thermofilum, or the genus Archaeoglobus.

10-11. (canceled)

12. The recombinant cell according to claim 1, further comprising a gene encoding carbon monoxide dehydrogenase as a second foreign gene.

13. The recombinant cell according to claim 1, wherein the first foreign gene is a gene encoding isoprene synthase, the recombinant cell produces isoprene, and the isoprene synthase is derived from a plant, and
the recombinant cell further comprising a gene encoding isopentenyl diphosphate isomerase as a third foreign gene.

14. The recombinant cell according to claim 13, wherein the isoprene synthase is a protein of the following (a-1), (a-2), or (a-3):
(a-1) a protein consisting of an amino acid sequence of SEQ ID NO: 1,
(a-2) a protein consisting of an amino acid sequence in which 1 to 20 amino acids are deleted, substituted, or added in the amino acid sequence of SEQ ID NO: 1, and having isoprene synthase activity, and
(a-3) a protein consisting of an amino acid sequence having identity of 90% or more with the amino acid sequence of SEQ ID NO: 1, and having isoprene synthase activity.

15. (canceled)

16. The recombinant cell according to claim 1, wherein the first foreign gene is a gene encoding sesquiterpene synthase, the recombinant cell produces terpene having 15 carbon atoms, and the sesquiterpene synthase is farnesene synthase.

17. The recombinant cell according to claim 1, wherein the first foreign gene is a gene encoding sesquiterpene synthase, the recombinant cell produces terpene having 15 carbon atoms, and the sesquiterpene synthase is cyclic sesquiterpene synthase.

18-20. (canceled)

21. The recombinant cell according to claim 1, wherein the first foreign gene is a gene encoding monoterpene synthase, the recombinant cell produces terpene having 10 carbon atoms, and the monoterpene synthase is cyclic monoterpene synthase.

22-31. (canceled)

32. The recombinant cell according to claim 1,
wherein the first foreign gene is a gene encoding squalene synthase, the recombinant cell produces terpene having 30 carbon atoms,
the recombinant cell further comprising a gene encoding isopentenyl diphosphate isomerase as a twelfth foreign gene.

33-35. (canceled)

36. The recombinant cell according to claim 1, further comprising at least one gene selected from the group consisting of a gene encoding acetoacetyl-CoA thiolase, a gene encoding HMG-CoA synthase, a gene encoding HMG-CoA reductase, a gene encoding mevalonate-5-kinase, a gene encoding mevalonate-5-phosphate decarboxylase, and a gene encoding isopentenyl phosphate kinase as a sixteenth foreign gene.

37. (canceled)

38. A method for producing isoprene or terpene, the method comprising bringing at least one carbon source selected from the group consisting of carbon monoxide, carbon dioxide, methane, methanol, and acetic acid into contact with the recombinant cell according to claim 1, thereby allowing the recombinant cell to produce isoprene or terpene having 10, 15, 20, 30, or 40 carbon atoms from the carbon source.

39. The method according to claim 38, the method comprising culturing the recombinant cell using at least one carbon source selected from the group consisting of carbon monoxide, carbon dioxide, methane, methanol, and acetic acid, and obtaining isoprene or terpene having 10, 15, 20, 30, or 40 carbon atoms from the cultured product.

40-45. (canceled)

46. A method for producing isoprene, the method comprising bringing at least one carbon source selected from the group consisting of carbon monoxide and acetic acid into contact with the recombinant cell according to claim 13, thereby allowing the recombinant cell to produce isoprene from the carbon source.

47. The method according to claim 46, the method comprising culturing the recombinant cell using at least one carbon source selected from the group consisting of carbon monoxide and acetic acid, and obtaining isoprene from the cultured product.
